SEQUENCE LISTING (6 sequences)

SEQ ID NO: 1 - DNA, 777 bp, Pseudomonas mendocina
gctcctcttcctgatacaccgggagcgccatttcctgctgtcgcaaacttcgaccgcagcggcccttacactgtttctagccagtcagaagggccgagctgtcgcatctatagacctcgcgacctgggtcagggaggcgtacgccatccggttattctttggggcaacggcactggtgctggaccgtctacatatgcaggcttgctttcacactgggcaagccacggtttcgttgtagcggctgcggaaacatctaacgctggtaccggacgcgaaatgctcgcctgcctggactatctggtacgtgagaacgacaccccctacggcacctattccggcaagctcaataccgggcgagtcggcacttctgggcattctcaaggtggaggcgggtcaatcatggctggccaggatacgagagtacgtacaacggcgccgatccagccttacactcttggcctgggacacgacagcgcttctcaacgccgccaacagggaccgatgttccttatgtctggtggcggagacacaatcgctttcccttacctcaacgctcagccggtctaccgccgtgcaaacgtacctgtattctggggcgaaagacgttacgtttcacacttcgaaccggtaggtagcggtggggcttatcgcggcccgtctacagcatggttccgcttccaacttatggatgaccaagacgctcgcgctacattctacggcgcgcagtgcagcctttgcacttctttactttggtcagtcgaacgccgcgggctttaa

SEQ ID NO: 2 - protein, 258 aa, Pseudomonas mendocina
APLPDTPGAPFPAVANFDRSGPYTVSSQSEGPSCRIYRPRDLGQGGVRHPVILWGNGTGAGPSTYAGLLSHWASHGFVVAAAETSNAGTGREMLACLDYLVRENDTPYGTYSGKLNTGRVGTSGHSQGGGGSIMAGQDTRVRTTAPIQPYTLGLGHDSASQRRQQGPMFLMSGGGDTIAFPYLNAQPVYRRANVPVFWGERRYVSHFEPVGSGGAYRGPSTAWFRFQLMDDQDARATFYGAQCSLCTSLLWSVERRGL

SEQ ID NO: 3 - DNA, 608 bp, Artificial
  aprE promoter DNA sequence
gaattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaaga

SEQ ID NO: 4 - DNA, 87 bp, Artificial
  aprE signal peptide DNA sequence
gtgagaagcaaaaaattgtggatcagcttgttgtttgcgttaacgttaatctttacgatggcgttcagcaacatgtctgcgcaggct

SEQ ID NO: 5 - DNA, 87 bp, Artificial
  hybrid aprE-P. mendocino lipase signal peptide DNA sequence
gtgagaagcaaaaaattgtggatcagcttgttgtttgcgttaacgttagcggcttcttgcctgagcgtctgtgcaactgtagctgca

SEQ ID NO: 6 - DNA, 247 bp, Artificial
  BPN′ terminator DNA sequence
acataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

(19) United States
(12) Patent Application Publication
(10) Pub. No.: US 2024/0240115 A1
(43) Pub. Date: Jul. 18, 2024

(54) COMPOSITION WITH IMPROVED MOISTURE MANAGEMENT PERFORMANCE

(71) Applicant: HENKEL AG & CO. KGAA, Düsseldorf (DE)

(72) Inventors: Christina ROELEKE, Mönchengladbach (DE); Shohana ISLAM, Düsseldorf (DE); Ramona JASNY, Mettmann (DE); Susanne WIELAND, Zons/Dormagen (DE); Christian DEGERING, Erkrath (DE); Karin KANIA, Mönchengladbach (DE); Hanka SCHROETER, Düsseldorf (DE); Martina SEILER, Duisburg (DE); Christian D. ADAMS, Palo Alto, CA (US); Lilia Maria BABE, Palo Alto, CA (US); Amy DEMING LIU, Palo Alto, CA (US)

(21) Appl. No.: 18/571,882

(22) PCT Filed: Jun. 27, 2022
(86) PCT No.: PCT/EP2022/067508

§ 371 Date: Dec. 19, 2023

Related U.S. Application Data
(60) Provisional application No. 63/216,552, filed on Jun. 30, 2021.

(51) Int. Cl. C11D 3/386; C11D 3/20; C11D 3/33; C11D 3/37; C12N 9/18; D06M 16/00

(52) U.S. Cl. CPC C11D 3/38636; C11D 3/2082; C11D 3/33; C11D 3/3769; C12N 9/18; C12Y 301/01074; D06M 16/003

(57) ABSTRACT

The present invention relates to cleaning and fabric conditioning compositions comprising at least one lipolytic enzyme having polyesterase activity and at least one additional ingredient. The compositions described herein are suitable for use in cleaning and fabric conditioning processes and include detergent compositions, such as laundry detergent compositions, in particular liquid laundry detergent compositions, and softener compositions. The present invention further relates to a method for cleaning or conditioning textiles and to the use of the cleaning composition according to the invention for improving the thermophysiological properties and/or increasing hydrophilicity of textiles and fabrics as well as the corresponding methods.

### FIELD OF THE INVENTION

The present invention relates to cleaning and fabric conditioning compositions comprising at least one lipolytic enzyme having polyesterase activity and at least one additional ingredient. The compositions described herein are suitable for use in cleaning and fabric conditioning processes and include detergent compositions, such as laundry detergent compositions, in particular liquid laundry detergent compositions, and softener compositions. The present invention further relates to a method for cleaning or conditioning textiles and to the use of the cleaning composition according to the invention for improving the thermophysiological properties and/or increasing hydrophilicity of textiles and fabrics as well as the corresponding methods.

### BACKGROUND

Enzymes have been used in detergents for decades, wherein most commercially relevant are the proteases and amylases effectively removing protein and starch related soiling, respectively. However, most household care related soiling is a complex mixture of various organic matters. Consequently, stain removal requires different enzyme activity, which vary depending on the specific stain targeted. Beside to cleaning effects of specific enzymes, there are usually further enzymes included, which relate to fabric care, e.g., anti-gray and/or anti-pilling.

Polyester has become more and more important for textile production over the recent years. It is known that polyester textiles suffer from the drawback of having comparably hydrophobic fiber surfaces. This has led to a lower wear comfort compared to, e.g., cotton, since polyester fibers have a much lower capacity to absorb moisture.

Various enzymes, such as lipolytic enzymes (also known as lipases), are able to catalyze the hydrolysis of a variety of polymers, including polyesters. Some of these enzymes are being investigated for use in a number of industrial applications, such as detergents for laundry and dishwashing applications. The use of such enzymes is of particular interest for hydrolyzing polyesters, such as polyethylene terephthalate (PET).

There is a continuing need for lipolytic enzymes with improved activity and/or improved stability that can be used in compositions for treating fabrics and/or textiles, in particular those comprising or consisting of polyesters. In particular there is need for solutions to increase the wear comfort of textiles comprising or consisting of polyester.

### SUMMARY OF THE INVENTION

Surprisingly, the inventors of the present invention have found that the lipolytic enzymes having polyesterase activity described herein are active under washing process conditions and have various nourishing properties for textiles consisting of or comprising polyester, such as polyethylene terephthalate (PET). This is surprising insofar as such enzymes known to date are more active at higher temperatures (>60° C.) and, moreover, are only able to degrade polyester/PET very slowly. However, the lipolytic enzyme having polyesterase activity used in the cleaning composition according to the present invention demonstrates rapid polyester degradation at 40° C. It was not only found that said enzyme prevents pilling on new polyester textiles, it can also produce what is referred to as a “renew” effect. The lipolytic enzyme having polyesterase activity also prevents the graying of white laundry and the fading/graying of colored laundry. It has also been found that, with the appropriate dosage, all of these positive washing properties can be achieved without significantly damaging the fiber. Because the textiles look new longer, they are worn longer and are replaced less quickly. This leads to a reduction in the CO₂ footprint, since less polyester is used.

The inventors have also surprisingly found that the lipolytic enzyme having polyesterase activity described herein also improves wear comfort of textiles and fabrics made of or comprising polyester in that it increases the property of the polyester fibers to absorb moisture.

Therefore, in a first aspect, the present invention is direct to a cleaning or fabric conditioning composition, comprising
(a) at least one variant lipolytic enzyme, wherein said variant lipolytic enzyme comprises an amino acid sequence having at least 70% identity to the full length amino acid sequence of SEQ ID NO:2, comprising the substitutions T064V-T117L-T177N/R-I178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161H, G175A/E, F207L/T, V210I, Q227H, A236P, S244E, E254Q, and R256K, wherein the positions are numbered by reference to the amino acid sequence of SEQ ID NO:2, and wherein the variant has polyesterase activity; and
(b) at least one additional ingredient selected from the group consisting of performance polymers, complexing agents, surfactants, and combinations thereof.

In various embodiments, the variant lipolytic enzyme comprises an amino acid sequence having at least 75%, 80%, 85%, 90%, 91%, 92%, 93%, 94%, 95%, 96%, 97%, 98%, or 99% identity to the full-length amino acid sequence of SEQ ID NO:2. In various embodiments, the variant lipolytic enzyme is derived from a parent enzyme comprising an amino acid sequence having at least 75%, 80%, 85%, 90%, 91%, 92%, 93%, 94%, 95%, 96%, 97%, 98%, or 99% identity to the full-length amino acid sequence of SEQ ID NO:2. In various embodiments, the variant lipolytic enzyme comprises a combination of substitutions selected from the group consisting of R40T-T64V-T117L-G175E-T177N-F180P-Y182A-R190L-S205G-F207L-S212D-F226L-Y239I-L249P-S252I-L258F, R40T-G61D-T64V-S70E-T117L-T177N-I178L-F180P-Y182A-R190L-S205G-F207T-S212D-F226L-Q227H-A236P-Y239I-L249P-S252I-E254Q-L258F, R40T-T64V-S70E-T117L-T177N-I178L-F180P-Y182A-R190L-S205G-F207T-S212D-F226L-A236P-Y239I-L249P-S252I-E254Q-L258F, R40A-T64V-S70E-T117L-T177N-I178L-F180P-Y182A-R190L-S205G-F207T-S212D-F226L-A236P-Y239I-L249P-S252I-E254Q-L258F, R40T-T64V-S70E-T117L-Q161H-T177N-I178L-F180P-Y182A-R190L-S205G-F207T-S212D-F226L-A236P-Y239I-L249P-S252I-E254Q-L258F, R40T-T64V-S70E-T117L-G175A-T177N-I178L-F180P-Y182A-R190L-S205G-F207T-S212D-F226L-A236P-Y239I-L249P-S252I-E254Q-L258F, R40T-T64V-S70E-T117L-T177N-I178L-F180P-Y182A-R190L-S205G-F207T-V210I-S212D-F226L-A236P-Y239I-L249P-S252I-E254Q-L258F, R40T-T64V-S70E-T117L-T177N-I178L-F180P-Y182A-R190L-S205G-F207T-S212D-F226L-A236P-Y239I-S244E-L249P-S252I-E254Q-L258F, R40T-T64V-S70E-T117L-T177N-I178L-F180P-Y182A-R190L-S205G-F207T-S212D-F226L-A236P-Y239I-L249P-S252I-E254Q-R256K-L258F, V14S-R40A-G59Y-G61D-T64V-A66D-S70E-T117L-Q161H-T177R-I178L-F180P-Y182A-R190L-S205G-F207T-V210I-S212D-F226L-A236P-Y239I-L249P-S252I-E254Q-R256K-L258F, V14S-R40A-G59Y-G61D-T64V-S70E-T117L-Q161H-T177R-I178L-F180P-Y182A-R190L-S205G-F207T-V210I-S212D-F226L-A236P-Y239I-L249P-S252T-E254Q-R256K-L258F, R40T-G61D-T64V-S70E-T117L-Q161H-T177R-1178L-F180P-Y182A-R190L-S205G-F207T-V210I-S212D-F226L-A236P-Y239I-L249P-S252I-E254Q-R256K-L258F, and V14S-R40A-G59Y-G61D-T64V-A66D-S70E-T117L-Q161H-G175A-T177R-I178L-F180P-Y182A-R190L-S205G-F207T-V210I-S212D-F226L-A236P-Y239I-L249P-S252I-E254Q-R256K-L258F, wherein the positions are numbered by reference to the amino acid sequence of SEQ ID NO:2.

In various embodiments, the variant lipolytic enzyme has lipolytic activity on a polyester selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof.

In various embodiments, the variant lipolytic enzyme is contained in the composition in an amount of from 0.00001 to 1 wt. %, preferably in an amount of from 0.0001 to 0.5 wt. %, particularly preferably in an amount of from 0.001 to 0.1 wt. %.

In various embodiments, the at least one additional ingredient comprises a performance polymer. The performance polymer may be an alkoxylated polyethylene imine. In various embodiments, the at least one additional ingredient comprises a complexing agent.

In various embodiments, the composition comprises at least one further ingredient selected from the group consisting of builders, bleaching agents, bleach activators, water-miscible organic solvents, sequestering agents, electrolytes, pH regulators, optical brighteners, graying inhibitors, foam regulators, dyes and fragrances and combinations thereof. In various embodiments, the cleaning or fabric conditioning composition has a pH of 7.0 to 11.0, as measured in 1 wt. % aqueous solution at 20° C. The cleaning or fabric conditioning composition may be present in solid or liquid form, for example in liquid form. In various embodiments, the cleaning or fabric conditioning composition is in unit dose form.

In another aspect, the present invention is directed to a method of cleaning or conditioning a textile or fabric, comprising:
a) providing a cleaning composition comprising at least one variant lipolytic enzyme, wherein said variant lipolytic enzyme comprises an amino acid sequence having at least 70% identity to the full length amino acid sequence of SEQ ID NO:2, comprising the substitutions T064V-T117L-T177N/R-I178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161H, G175A/E, F207L/T, V210I, Q227H, A236P, S244E, E254Q, and R256K, wherein the positions are numbered by reference to the amino acid sequence of SEQ ID NO:2, and wherein the variant has polyesterase activity; and at least one additional ingredient selected from the group consisting of complexing agents, surfactants, performance polymers and combinations thereof; and
b) contacting the textile or fabric with the composition, wherein the textile or fabric comprises or consists of polyester, and wherein the polyester is preferably selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof, more preferably selected from the group consisting of polyethylene terephthalate (PET), polytrimethylene terephthalate (PTT), polybutylene terephthalate (PBT), and combinations thereof.

In still another aspect, the invention relates to a method for improving the thermophysiological properties of a textile or fabric comprising or consisting of polyester, the method comprising
a) providing a cleaning composition comprising at least one variant lipolytic enzyme, wherein said variant lipolytic enzyme comprises an amino acid sequence having at least 70% identity to the full length amino acid sequence of SEQ ID NO:2, comprising the substitutions T064V-T117L-T177N/R-1178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161H, G175A/E, F207L/T, V210L, Q227H, A236P, S244E, E254Q, and R256K, wherein the positions are numbered by reference to the amino acid sequence of SEQ ID NO:2, and wherein the variant has polyesterase activity; and
b) contacting the fabric or textile with the composition.

In various embodiments, the thermophysiological properties comprise heat and moisture management, wear comfort or any combination thereof.

In still another aspect, the invention is directed to a method for increasing the hydrophilicity of a textile or fabric comprising or consisting of polyester, the method comprising
a) providing a cleaning composition comprising at least one variant lipolytic enzyme, wherein said variant lipolytic enzyme comprises an amino acid sequence having at least 70% identity to the full length amino acid sequence of SEQ ID NO:2, comprising the substitutions T064V-T117L-T177N/R-I178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161H, G175A/E, F207L/T, V210I, Q227H, A236P, S244E, E254Q, and R256K, wherein the positions are numbered by reference to the amino acid sequence of SEQ ID NO:2, and wherein the variant has polyesterase activity; and
b) contacting the fabric or textile with the composition.

In various embodiments of the methods described herein, the polyester is preferably selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof, more preferably selected from the group consisting of polyethylene terephthalate (PET), polytrimethylene terephthalate (PTT), polybutylene terephthalate (PBT), and combinations thereof.

### DETAILED DESCRIPTION

### Definitions

When in the following reference is made to the enzyme according to the invention, the terms “lipolytic enzyme”, “variant lipolytic enzyme”, “lipolytic enzyme having polyesterase activity” or “polyesterase” (or the like) are meant to be used equivalently.

Such enzymes used in cleaning compositions according to the invention are characterized by having polyester degrading activity as described herein.

In the context of the present invention an enzyme having “polyesterase activity” refers to an enzyme that has significant capability to catalyze the hydrolysis and/or surface modification of polyester as described herein.

If not indicated otherwise, all references to percentages in relation to the compositions disclosed herein relate to wt. % relative to the total weight of the respective composition. It is understood that when reference is made to compositions that comprise enzymes as defined herein, the respective composition comprises at least one of each of the specified enzymes but can also comprise two or more of each enzyme type, such as two or more lipolytic enzymes having polyesterase activity (polyesterases).

Unless defined otherwise herein, all technical and scientific terms used herein have the same meaning as commonly understood by one of ordinary skill in the art to which this invention pertains. Although any methods and materials similar or equivalent to those described herein find use in the practice of the present invention, the preferred methods and materials are described herein. Accordingly, the terms defined immediately below are more fully described by reference to the specification as a whole. Also, as used herein, the singular terms “a,” “an,” and “the” include the plural reference unless the context clearly indicates otherwise. It is to be understood that this invention is not limited to the particular methodology, protocols, and reagents described herein, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

When in the following reference is made to the compositions according to the invention, the terms “cleaning composition”, “detergent composition”, “laundry detergent composition”, “laundry detergent”, “detergent”, “washing agent” or “agent” (or the like) are meant to be understood to be used equivalently. As used herein, the term “detergent composition” or “cleaning composition”, includes unless otherwise indicated, granular or powder-form all-purpose, light-duty washing agents or heavy-duty washing agents, especially cleaning detergents; liquid, gel or paste-form all-purpose washing agents, especially the so-called heavy-duty liquid (HDL) and light-duty liquid (LDL) types; liquid fine-fabric detergents; as well as cleaning auxiliaries such as bleach additives and “stain-stick” or pre-treat types. The terms “detergent composition” and “detergent formulation” are used in reference to mixtures which are intended for use in a wash medium for the cleaning of soiled objects. In some aspects, the term is used in reference to laundering fabrics and/or garments (e.g., “laundry detergents”). The terms “fabric conditioning composition”, “fabric finisher” or “fabric care composition”, as interchangeably used herein, are meant to include all compositions that are used to impart certain properties to fabrics and textiles treated therewith, such as softeners, anti-wrinkle compositions, perfuming compositions and the like. It is not intended that the present invention be limited to any particular detergent formulation or composition, unless otherwise indicated by the definition provided herein. The term “detergent composition” is not intended to be limited to compositions that comprise surfactants. It is intended that in addition to the variants according to the invention, the term encompasses detergents that may comprise, e.g., surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxidoreductases, bluing agents and fluorescent dyes, anti-oxidants, and solubilizers.

As used herein, the term “cotton” refers to refers to Gossypium hirsutum and includes all plant varieties that can be bred with cotton, including wild cotton species as well as those plants belonging to Gossypium that permit breeding between species. In the context of the invention, the term “cotton” does not only relate to the plant Gossypium but also to the cellulosic material derived from the plant which can be processed to fibers, yarns, fabrics, textiles and the like as pertinent to the person skilled in the art.

As used herein, the term “effective amount of enzyme” refers to the quantity of enzyme necessary to achieve the enzymatic activity required in the specific application, e.g., in a defined detergent composition. Such effective amounts are readily ascertained by one of ordinary skill in the art and are based on many factors, such as the particular enzyme used, the cleaning application, the specific composition of the detergent composition, and whether a liquid or dry (e.g., granular, bar) composition is required, and the like.

As used herein, the term “fabric” encompasses any textile material. Thus, it is intended that the term encompass garments, as well as fabrics, yarns, fibers, filaments, woven materials, non-woven materials, knit materials, natural materials, synthetic materials, and any other textile material.

The term “graying” or “greying” as used herein refers to the detachment and reattachment of dirt to a textile during a wash cycle. The term “anti-gray performance” or “anti-graying effect” (or the like) as used herein, therefore, refers to keeping the dirt that has been detached from the fiber during the washing of textiles suspended in the liquor, thus preventing the dirt from being reattached to the textile. Such performance can be determined by methods known in the prior art. For example, textile from a test wash and reference textiles may be examined and evaluated by visual inspection. Alternatively, an evaluation may be made, e.g., by the absorption or extinction of light measured by an appropriate detector (e.g., photometer). In preferred embodiments of the invention, anti-graying performance means that textiles washed with a detergent under test exhibit no more than 90%, no more than 80%, no more than 70%, no more than 60%, no more than 50%, no more than 40%, no more than 30%, no more than 20%, no more than 10%, or no more than 5% of the graying exhibited by textiles washed with a comparable reference detergent (e.g., without cellulase and/or without polyesterase or containing a known reference cellulase and/or polyesterase).

The term “wear comfort”, as used herein, is meant to refer to the comfort experienced when wearing a textile treated with the compositions described herein. This term includes thermophysiological properties such as heat and moisture management that may be influenced by the hydrophilicity of the textile fibers. “Heat management”, as used in this context, refers to the ability to effectively exchange heat, for example body heat produced by the wearer, and at the same time provide thermal insulation. Similarly, “moisture management”, as used herein, refers to the ability to soak up moisture and release it again. “Hydrophilicity”, as used herein, relates to the property to attract water and thus to the wetting properties of a surface. Highly hydrophilic surfaces are easily wetted by water, while highly hydrophobic surfaces repel water and thus are not easily wetted.

As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes).

As used herein, the term “laundering” includes both household laundering and industrial laundering and means the process of treating textiles with a solution comprising a cleaning or detergent composition as provided herein. The laundering process can be carried out using, e.g., a household or an industrial washing machine or can be carried out by hand. Such “laundering” is also included in the term “cleaning”, as used herein. “Fabric conditioning” includes all types of processes of treating textiles with a solution comprising a fabric conditioning composition as provided herein. Such treating can occur in a household or an industrial washing machine or can be carried out by hand.

As used herein, the term “mature polypeptide” means a polypeptide in its final form following translation and any post-translational modifications, such as N-terminal processing, C-terminal truncation, glycosylation, phosphorylation, etc.

As used herein, the term “polyamide” refers to synthetic polymers like polyamide 6.6 (PA 6.6; nylon) which is formed from hexamethylene diamine and adipic acid through polycondensation and polyamide 6 (PA 6; perlon) which is made from caprolactam through polymerization.

As used herein, the term “polyester-containing material” or “polyester-containing product” refers to a product, such as a textile, fabric, or plastic product, comprising at least one polyester in crystalline, semi-crystalline, or substantially amorphous forms. In some embodiments, the polyester-containing material refers to a textile or fabric or fibers comprising at least one polyester. In some embodiments, the polyester-containing material refers to a textile or fabric or fibers consisting of at least one polyester. In some embodiments, the polyester-containing material refers to a textile or fabric or fibers comprising besides at least one polyester further component(s), like e.g., cellulosic materials or polyamides or synthetic polymers. In some embodiments, the polyester-containing material refers to a textile or fabric or fibers comprising at least one polyester and at least one cellulosic material, in particular relating to, e.g., cotton-polyester blends.

As used herein, the term “polyester” refers to its monomer bonded by ester linkage. As used herein, the term “polyester” includes, but is not limited to, those polyesters selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof.

As used herein, the term “polymer” refers to a chemical compound or mixture of compounds whose structure is constituted of multiple repeating units linked by covalent chemical bonds. Within the context of the disclosure, the term polymer includes natural or synthetic polymers, constituting of a single type of repeat unit (i.e., homopolymers) or of a mixture of different repeat units (i.e., block copolymers and random copolymers).

As used herein, the term “textile” refers to any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, e.g., denims and toweling non-wovens, felts, yarns. The textile may include cellulose based such as natural cellulosics, including cotton, flax/linen, jute, ramie, sisal or coir or manmade cellulosics (e.g., originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as polyamide, e.g., nylon, perlon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g., polyamide fiber, acrylic fiber, polyester fiber, polyvinyl chloride fiber, polyurethane fiber, polyurea fiber, aramid fiber), and/or cellulose-containing fiber (e.g., rayon/viscose, ramie, flax/linen, jute, cellulose acetate fiber, lyocell). Fabric may be conventional washable laundry, e.g., stained household laundry. When the term fabric or garment is used, it is intended to include the broader term textiles as well. In the context of the present application, the term “textile” is used interchangeably with fabric and cloth. In some embodiments, textiles include those materials that include at least one polyester.

As used herein, the term “variant polypeptide” refers to a polypeptide comprising an amino acid sequence that differs in at least one amino acid residue from the amino acid sequence of a parent or reference polypeptide (including but not limited to wild-type polypeptides). In some embodiments, the parent polypeptide for use herein comprises an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:2.

As used herein, the term “viscose” refers to in general cellulosic man-made fibers, which are natural cellulose polymers extracted from plants and produced in a spinnable solution via viscose process.

As used herein, the term “wash cycle” refers to a washing operation in which textiles are immersed in a wash liquor, mechanical action of some kind is applied to the textile to release stains or to facilitate flow of wash liquor in and out of the textile and finally the superfluous wash liquor is removed. After one or more wash cycles, the textile is generally rinsed and dried.

As used herein, the term “wash liquor” refers to the solution or mixture of water and detergent components optionally including variant lipolytic enzymes as provided herein.

### Variant Lipolytic Enzymes Having Polyesterase Activity

The present invention relates to cleaning compositions comprising novel variant lipolytic enzymes having hydrolytic activity on at least one polyester. In particular, the present invention relates to cleaning compositions comprising variant lipolytic enzymes having polyesterase activity (polyesterases). The present invention relates to cleaning compositions comprising lipolytic enzymes, which are esterases. The present invention relates to cleaning compositions comprising lipolytic enzymes, which are polyesterases (lipolytic enzyme having polyesterase activity).

As used herein, a “lipase”, “lipase enzyme”, “lipolytic enzyme”, “lipolytic polypeptide”, or “lipolytic protein” is an enzyme, polypeptide, or protein exhibiting a lipid degrading capability such as a capability of degrading a triglyceride or a phospholipid. The lipolytic enzyme can be, e.g., a lipase, a phospholipase, an esterase or a cutinase. Lipolytic enzymes can be enzymes having a/P hydrolase fold. These enzymes typically have a catalytic triad of serine, aspartic acid and histidine residues. The a/P hydrolases include lipases and cutinases. Cutinases show little, if any, interfacial activation, where lipases often undergo a conformational change in the presence of a lipid-water interface. An active fragment of a lipolytic enzyme is a portion of a lipolytic enzyme that retains a lipid degrading capability. An active fragment retains the catalytic triad. As used herein, lipolytic activity can be determined according to any procedure known in the art (e.g., Gupta et al., Biotechnol. Appl. Biochem. 37: 63-71, 2003; U.S. Pat. No. 5,990,069; WO 96/18729). In one embodiment, lipolytic activity can be determined on 4-nitrophenyl butyrate (pNB) as provided in example 2.

As used herein, “cutinase” refers to lipolytic enzymes capable of hydrolyzing cutin substrates. Cutinases include those derived from various fungi and from bacterial sources. Cutinases may be naturally occurring or genetically modified cutinase obtained by UV irradiation, N-methyl-N′-nitroso guanidine (NTG) treatment, ethyl methane sulfonate (EMS) treatment, nitrous acid treatment, acridine treatment or the like, recombinant strains induced by the genetic engineering procedures such as cell fusion and gene recombination and so forth.

As used herein, the term “lipolytic enzyme having polyesterase activity” or “polyesterase” or “PETase” refers to an enzyme that has significant capability to catalyze the hydrolysis and/or surface modification of polyester. Suitable polyesterases may be isolated from animal, plant, fungal and bacterial sources. The aforementioned microorganisms may be, in addition to being isolated from wild strains, may be isolated from any of mutant strains obtained by UV irradiation, N-methyl-N′-nitroso guanidine (NTG) treatment, ethyl methane sulfonate (EMS) treatment, nitrous acid treatment, acridine treatment or the like, recombinant strains induced by the genetic engineering procedures such as cell fusion and gene recombination and so forth. The polyesterase may catalyze the hydrolysis and/or surface modification of a polyester selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof.

As used herein, “% identity or percent identity” refers to sequence similarity. Percent identity may be determined using standard techniques known in the art (e.g., Smith and Waterman, Adv. Appl. Math. 2: 482, 1981; Needleman and Wunsch, J. Mol. Biol. 48: 443, 1970; Pearson and Lipman, Proc. Natl. Acad. Sci. USA 85: 2444, 1988; software programs such as GAP, BESTFIT, FASTA, and TFASTA in the Wisconsin Genetics Software Package (Genetics Computer Group, Madison, WI); and Devereux et al., Nucl. Acid Res. 12: 387-395, 1984).

As used herein, “homologous proteins”, “homologs” or “homologous proteins” refers to proteins that have distinct similarity in primary, secondary, and/or tertiary structure. Protein homology can refer to the similarity in linear amino acid sequence when proteins are aligned. Homology can be determined by amino acid sequence alignment, e.g., using a program such as BLAST, MUSCLE, or CLUSTAL. Homologous search of protein sequences can be done using BLASTP and PSI-BLAST from NCBI BLAST with threshold (E-value cut-off) at 0.001 (Altschul et al., Nucleic Acids Res, 25 (17): 3389-402, 1997). One example of a useful algorithm is PILEUP. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (Feng and Doolittle, J. Mol. Evol. 35: 351-360, 1987). The method is similar to that described by Higgins and Sharp (Higgins and Sharp, CABIOS 5: 151-153, 1989). Other useful algorithm is the BLAST algorithms described by Altschul et al., (Altschul et al., J. Mol. Biol. 215: 403-410, 1990; Karlin and Altschul, Proc. Natl. Acad. Sci. USA 90: 5873-5787, 1993). The BLAST program uses several search parameters, most of which are set to the default values. Amino acid sequences can be entered in a program such as the Vector NTI Advance suite and a Guide Tree can be created using the Neighbor Joining (NJ) method (Saitou and Nei, Mol Biol Evol, 4: 406-425, 1987). The tree construction can be calculated using Kimura's correction for sequence distance and ignoring positions with gaps. A program such as AlignX can display the calculated distance values in parentheses following the molecule name displayed on the phylogenetic tree. The CLUSTAL W algorithm is another example of a sequence alignment algorithm (Thompson et al., Nucleic Acids Res, 22: 4673-4680, 1994).

A percent (%) amino acid sequence identity value is determined by the number of matching identical residues divided by the total number of residues of the “reference” sequence including any gaps created by the program for optimal/maximum alignment. If a sequence is 90% identical to SEQ ID NO:A, SEQ ID NO:A is the “reference” sequence. BLAST algorithms refer the “reference” sequence as “query” sequence.

In some embodiments, the variant lipolytic enzymes used in compositions according to the invention comprise an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:2. In some embodiments, the variant lipolytic enzymes used in compositions according to the invention have an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:2 and has esterase activity.

In one embodiment, the variant lipolytic enzymes used in compositions according to the invention comprise an amino acid sequence having at least 70% identity to the full length amino acid sequence of SEQ ID NO:2, comprising the substitutions X064V-X117L-X177N/R-X178L-X180P-X182A-X190L-X205G-X212D-X226L-X239I-X249P-X252I-X258F, and further comprising at least one additional substitution selected from the group consisting of X014S, X040A/T, X059Y, X061D, X066D, X070E, X161H, X175A/E, X207L/T, X210I, X227H, X236P, X244E, X254Q, and X256K, where the positions are numbered by reference to the amino acid sequence of SEQ ID NO:2, and where the variant has esterase activity.

In some embodiments, the variant lipolytic enzymes used in compositions according to the invention comprise an amino acid sequence having at least 70% identity to the full length amino acid sequence of SEQ ID NO:2, comprising the substitutions T064V-T117L-T177N/R-I178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161H, G175A/E, F207L/T, V210I, Q227H, A236P, S244E, E254Q, and R256K, where the positions are numbered by reference to the amino acid sequence of SEQ ID NO:2, and where the variant has esterase activity.

In some embodiments, the variant lipolytic enzymes used in compositions according to the invention comprise an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to the full length amino acid sequence of SEQ ID NO:2 comprising the substitutions T064V-T117L-T177N/R-I178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161H, G175A/E, F207TL/T, V210I, Q227H, A236P, S244E, E254Q, and R256K, where the positions are numbered by reference to the amino acid sequence of SEQ ID NO:2, and where the variant has esterase activity.

In some embodiments, the variant lipolytic enzymes used in compositions according to the invention comprise an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to the full length amino acid sequence of SEQ ID NO:2 and comprises a combination of mutations selected from the group consisting of R40T-T64V-T117L-G175E-T177N-F180P-Y182A-R190L-S205G-F207L-S212D-F226L-Y239I-L249P-S252I-L258F, R40T-G61D-T64V-S70E-T117L-T177N-I178L-F180P-Y182A-R190L-S205G-F207T-S212D-F226L-Q227H-A236P-Y239I-L249P-S252I-E254Q-L258F, R40T-T64V-S70E-T117L-T177N-I178L-F180P-Y182A-R190L-S205G-F207T-S212D-F226L-A236P-Y239I-L249P-S252I-E254Q-L258F, R40A-T64V-S70E-T117L-T177N-I178L-F180P-Y182A-R190L-S205G-F207T-S212D-F226L-A236P-Y239I-L249P-S252I-E254Q-L258F, R40T-T64V-S70E-T117L-Q161H-T177N-I178L-F180P-Y182A-R190L-S205G-F207T-S212D-F226L-A236P-Y239I-L249P-S252I-E254Q-L258F, R40T-T64V-S70E-T117L-G175A-T177N-I178L-F180P-Y182A-R190L-S205G-F207T-S212D-F226L-A236P-Y239I-L249P-S252I-E254Q-L258F, R40T-T64V-S70E-T117L-T177N-I178L-F180P-Y182A-R190L-S205G-F207T-V210I-S212D-F226L-A236P-Y239I-L249P-S252I-E254Q-L258F, R40T-T64V-S70E-T117L-T177N-I178L-F180P-Y182A-R190L-S205G-F207T-S212D-F226L-A236P-Y239I-S244E-L249P-S252I-E254Q-L258F, R40T-T64V-S70E-T117L-T177N-I178L-F180P-Y182A-R190L-S205G-F207T-S212D-F226L-A236P-Y239I-L249P-S252I-E254Q-R256K-L258F, V14S-R40A-G59Y-G61D-T64V-A66D-S70E-T117L-Q161H-T177R-I178L-F180P-Y182A-R190L-S205G-F207T-V210I-S212D-F226L-A236P-Y239I-L249P-S252I-E254Q-R256K-L258F, V14S-R40A-G59Y-G61D-T64V-S70E-T117L-Q161H-T177R-I178L-F180P-Y182A-R190L-S205G-F207T-V210I-S212D-F226L-A236P-Y239I-L249P-S252I-E254Q-R256K-L258F, R40T-G61D-T64V-S70E-T117L-Q161H-T177R-I178L-F180P-Y182A-R190L-S205G-F207T-V210I-S212D-F226L-A236P-Y239I-L249P-S252I-E254Q-R256K-L258F, and V14S-R40A-G59Y-G61D-T64V-A66D-S70E-T117L-Q161H-G175A-T177R-I178L-F180P-Y182A-R190L-S205G-F207T-V210I-S212D-F226L-A236P-Y239I-L249P-S252I-E254Q-R256K-L258F, where the positions are numbered by reference to the amino acid sequence of SEQ ID NO:2, and where the variant has esterase activity.

In some embodiments, the variant lipolytic enzymes used in compositions according to the invention comprise an amino acid sequence having at least 90% sequence identity to the full length amino acid sequence of SEQ ID NO:2 and comprises a combination of mutations selected from the group consisting of R40T-T64V-T117L-G175E-T177N-F180P-Y182A-R190L-S205G-F207L-S212D-F226L-Y239I-L249P-S252I-L258F, R40T-G61D-T64V-S70E-T117L-T177N-T178L-F180P-Y182A-R190L-S205G-F207T-S212D-F226L-Q227H-A236P-Y239I-L249P-S252I-E254Q-L258F, R40T-T64V-S70E-T117L-T177N-I178L-F180P-Y182A-R190L-S205G-F207T-S212D-F226L-A236P-Y239I-L249P-S252I-E254Q-L258F, R40A-T64V-S70E-T117L-T177N-I178L-F180P-Y182A-R190L-S205G-F207T-S212D-F226L-A236P-Y239I-L249P-S252I-E254Q-L258F, R40T-T64V-S70E-T117L-Q161H-T177N-I178L-F180P-Y182A-R190L-S205G-F207T-S212D-F226L-A236P-Y239I-L249P-S252I-E254Q-L258F, R40T-T64V-S70E-T117L-G175A-T177N-I178L-F180P-Y182A-R190L-S205G-F207T-S212D-F226L-A236P-Y239I-L249P-S252I-E254Q-L258F, R40T-T64V-S70E-T117L-T177N-I178L-F180P-Y182A-R190L-S205G-F207T-V210I-S212D-F226L-A236P-Y239I-L249P-S252I-E254Q-L258F, R40T-T64V-S70E-T117L-T177N-I178L-F180P-Y182A-R190L-S205G-F207T-S212D-F226L-A236P-Y239I-S244E-L249P-S252I-E254Q-L258F, R40T-T64V-S70E-T117L-T177N-I178L-F180P-Y182A-R190L-S205G-F207T-S212D-F226L-A236P-Y239I-L249P-S252I-E254Q-R256K-L258F, V14S-R40A-G59Y-G61D-T64V-A66D-S70E-T117L-Q161H-T177R-I178L-F180P-Y182A-R190L-S205G-F207T-V210I-S212D-F226L-A236P-Y239I-L249P-S252I-E254Q-R256K-L258F, V14S-R40A-G59Y-G61D-T64V-S70E-T117L-Q161H-T177R-I178L-F180P-Y182A-R190L-S205G-F207T-V210I-S212D-F226L-A236P-Y239I-L249P-S252I-E254Q-R256K-L258F, R40T-G61D-T64V-S70E-T117L-Q161H-T177R-I178L-F180P-Y182A-R190L-S205G-F207T-V210I-S212D-F226L-A236P-Y239I-L249P-S252I-E254Q-R256K-L258F, and V14S-R40A-G59Y-G61D-T64V-A66D-S70E-T117L-Q161H-G175A-T177R-I178L-F180P-Y182A-R190L-S205G-F207T-V210I-S212D-F226L-A236P-Y239I-L249P-S252I-E254Q-R256K-L258F, where the positions are numbered by reference to the amino acid sequence of SEQ ID NO:2, and where the variant has esterase activity.

In some embodiments, the variant lipolytic enzymes used in compositions according to the invention have esterase activity (e.g., ability to catalyze the hydrolysis and/or surface modification) on at least one polyester selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof. In one embodiment, the variant lipolytic enzymes provided herein have esterase activity on PET.

In a further embodiment of the invention, the polyesterase used in compositions according to the invention is characterized in that its anti-pilling performance is not significantly reduced compared to that of a polyesterase which comprises an amino acid sequence that corresponds to (or consists of) the amino acid sequence given in SEQ ID NO:2, i.e., has at least 70%, 75%, 80%, 85%, 90%, 95% of the reference anti-pilling performance. This relates in particular to variants which have the sequence identities or homologies given above.

In a further embodiment of the invention, the polyesterase used in compositions according to the invention is characterized in that its anti-graying performance is not significantly reduced compared to that of a polyesterase which comprises an amino acid sequence that corresponds to (or consists of) the amino acid sequence given in SEQ ID NO:2, i.e., has at least 70%, 75%, 80%, 85%, 90%, 95% of the reference anti-graying performance. This relates in particular to variants which have the sequence identities or homologies given above.

In a further embodiment of the invention, the polyesterase used in compositions according to the invention is characterized in that its performance with respect to heat and moisture management of treated fabrics/textiles is not significantly reduced compared to that of a polyesterase which comprises an amino acid sequence that corresponds to (or consists of) the amino acid sequence given in SEQ ID NO:2, i.e., has at least 70%, 75%, 80%, 85%, 90%, 95% of the reference anti-graying performance. This relates in particular to variants which have the sequence identities or homologies given above.

In a further embodiment of the invention, the polyesterase used in compositions according to the invention is characterized in that its anti-pilling and/or anti-graying and/or heat and moisture management performance is not significantly reduced compared to that of a polyesterase which comprises an amino acid sequence that corresponds to (or consists of) the amino acid sequence given in SEQ ID NO:2, i.e., has at least 70%, 75%, 80%, 85%, 90%, 95% of the reference anti-pilling performance and/or of reference anti-graying performance. This relates in particular to variants which have the sequence identities or homologies given above.

The anti-pilling and/or anti-graying performance can be determined in a washing system which comprises a washing agent in a dosage of between 4.5 and 7.0 g/L of washing liquor and the polyesterase, the polyesterases to be compared being used in the same concentration (based on active protein) and the anti-pilling and/or anti-graying performance being determined as described herein. For example, the washing operation can take place for 60 min at 40° C. and the water can have a water hardness between 15.5 and 16.5° dH (German hardness). The concentration of the polyesterase in the washing agent intended for this washing system is from 0.00001 to 1 wt. %, preferably from 0.0001 to 0.5 wt. %, particularly preferably from 0.001 to 0.1 wt. %, based on active protein.

The heat and moisture management performance can be determined in a washing system which comprises a washing agent in a dosage of between 4.5 and 7.0 g/L of washing liquor and the polyesterase, the polyesterases to be compared being used in the same concentration (based on active protein) and the heat and moisture management being determined by means of the absorbency of the textile fibers (wicking test) as described herein. For example, the washing operation can take place for 60 min at 40° C. and the water can have a water hardness between 15.5 and 16.5° dH (German hardness). The concentration of the polyesterase in the washing agent intended for this washing system is from 0.00001 to 1 wt. %, preferably from 0.0001 to 0.5 wt. %, particularly preferably from 0.001 to 0.1 wt. %, based on active protein.

A liquid washing agent for such a washing system may be composed as follows (in wt. %): 5-30% anionic surfactants, 0-5% amphoteric surfactants, 1-15% non-ionic surfactants, 0-4% citric acid/citrate, 0-5% performance polymers, 1-20% solvents in particular organic solvents, such as propane diol or ethanol, 0-1.5% boric acid, 0.0001-1% enzyme mix (e.g., protease, amylase, mannanase), 0.2-2% complex builders, minors (anti-foam, optical brightener, dye, perfume), and the remainder being demineralized water. Preferably, the dosage of the liquid washing agent is between 4.5 and 6.0 g/L of washing liquor, e.g., 4.7, 4.9 or 5.9 g/L of washing liquor. Washing preferably takes place in a pH range between pH 8 and pH 10.5, preferably between pH 8 and pH 9, as measured in 1 wt. % aqueous solution at 20° C.

Another preferred liquid washing agent for such a washing system is composed as follows (in wt. %): 2-6% anionic surfactants, 0.5-3% C₁₂₋₁₈ Na salts of fatty acids, 3-7% non-ionic surfactants, 0.1-2% phosphonates, 0.1-2% citric acid, 0.3-1% NaOH, 0.3-2% glycerol, 0.05-0.1% preservatives, 0.5-2% enzyme mix (e.g., protease, amylase, mannanase), minors (anti-foam, ethanol, dye, perfume), and the remainder being demineralized water. Preferably, the dosage of the liquid washing agent is between 4.5 and 6.0 g/L of washing liquor, e.g., 4.7, 4.9 or 5.9 g/L of washing liquor. Washing preferably takes place in a pH range between pH 8 and pH 10.5, preferably between pH 8 and pH 9, as measured in 1 wt. % aqueous solution at 20° C.

Another preferred liquid washing agent for such a washing system is composed as follows (in wt. %): 4.4% alkyl benzene sulfonic acid, 5.6% anionic surfactants, 2.4% C₁₂-C₁₈ Na salts of fatty acids, 4.4% non-ionic surfactants, 0.2% phosphonates, 1.4% citric acid, 0.95% NaOH, 0.01% defoamer, 2% glycerol, 0.08% preservatives, 1% ethanol, 1.6% enzyme mix (e.g., protease, amylase, mannanase) and the remainder being demineralized water. Preferably, the dosage of the liquid washing agent is between 4.5 and 6.0 g/L of washing liquor, e.g., 4.7, 4.9 or 5.9 g/L of washing liquor. Washing preferably takes place in a pH range between pH 8 and pH 10.5, preferably between pH 8 and pH 9, as measured in 1 wt. % aqueous solution at 20° C.

In the context of the invention, the anti-pilling and/or anti-graying performance is determined at 40° C. using a liquid washing agent as indicated above, the washing operation preferably taking place for 60 min.

In the context of the invention, the moisture management performance is determined at 28° C. according to DIN 53924 using a liquid washing agent as indicated above, the washing operation preferably taking place with water of a hardness of 6.7° dH.

The anti-pilling performance can be tracked using visual matching. In this case, a group of testers assigns the laundry to be examined a value on a scale of 1-5. The value=1 stands for very heavily pilled laundry, while the value=5 is assigned to un-pilled laundry.

The anti-graying performance can be tracked by measuring the Tristimulus-value (Y without UV) of the soiled fabric before and after wash with a spectrophotometer (Spectraflash SF600: without UV, Filter 420 nm, without brilliance). The higher the Y value, the higher is the re-whitening of pre-grayed textiles.

The moisture management, i.e. water absorbency, can be determined by measuring the height of capillary water in mm after 10 minutes. The higher the height of the capillary water in the fibers, the higher the absorbency and as a result the moisture management.

The activity-equivalent use of the relevant polyesterase ensures that the respective enzymatic properties, e.g., the anti-pilling and/or anti-graying performance and/or moisture management, are likened even if the ratio of active substance to total protein (the values of the specific activity) diverges. In general, a low specific activity can be compensated for by adding a larger amount of protein.

In a preferred embodiment, the composition of the invention comprises a variant lipolytic enzyme that has one or more improved properties when compared to a parent or reference lipolytic enzyme, wherein the improved property is selected from improved stability, improved hydrolytic activity on a polyester, or combinations thereof. In particular the improved property of the variant lipolytic enzyme is: (i) improved stability, wherein said variant has a residual activity at least 5% when measured in accordance with the stability assay of Example 3 and/or (ii) improved hydrolytic activity on a polyester, wherein said variant has a PI≥1.2 compared to the lipolytic enzyme having the amino acid sequence of SEQ ID NO:2 having the substitutions R40T-T64V-T117L-T177N-I178L-F180P-Y182A-R190L-S205G-F207T-S212D-F226L-Y239I-L249P-S252I-L258F when measured in accordance with the PET assay of Example 2.

### Polyester

The term “polyester”, as used herein, include polymers that contain at least one ester repeating unit in their main chain polymers. In their simplest form, polyesters are produced by polycondensation reaction of a glycol (diol) with a dicarboxylic acid (diacid) or its diester. Polyesters include naturally occurring chemicals, such as in the cutin of plant cuticles, as well as synthetics through step-growth polymerization such as polybutyrate.

Polyesters that can be contacted with the lipolytic enzymes having polyesterase activity described herein or a composition including such lipolytic enzymes having polyesterase activity include any ester bond-containing polymer. Such polyesters include aliphatic and aromatic polyesters. The aliphatic polyesters include: polyhydroxy alkanoates (PHA), which can be divided into polyhydroxy butyrate (PHB), polyhydroxy valerate (PHV), polyhydroxy hexanoate (PHH), and their copolymers; polylactide (PLA); poly-(F-caprolactone) (PCL); polybutylene succinate (PBS) and its derivative poly-(butylene succinate adipate) (PBSA). The aromatic polyesters include modified poly-(ethylene terephthalate) (PET) such as poly-(butylene adipate/terephthalate) (PBAT) and poly-(tetramethylene adipate-coterephthalate) (PTMAT); and aliphatic-aromatic copolyesters (AAC). In some embodiments, polyesters may be partially or substantially biodegradable.

In some embodiments, the polyesters may be partially or substantially resistant to microbial and enzymatic attack. In some embodiments, a polyester may be an aliphatic polyester. In some embodiments, a polyester may be an aromatic polyester. In some embodiments, an aromatic polyester may be a polyethylene terephthalate (PET). In some embodiments, an aromatic polyester may be a polytrimethylene terephthalate (PTT).

In some embodiments, the fabrics or textiles that find use in the methods according to the invention include fabrics and textiles that contain at least one polyester selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly-(ethylene adipate) (PEA), and combinations thereof.

In some embodiments, the invention provides methods for treating a fabric or a textile comprising contacting a fabric or a textile with a variant lipolytic enzyme having polyesterase activity described herein, or a composition comprising such variant lipolytic enzyme having polyesterase activity and optionally rinsing the fabric or textile.

In some embodiments, the contacting steps of the methods according to the invention comprise a variant lipolytic enzyme having polyesterase activity in an amount selected from the group consisting of 0.002 to 10000 mg of protein, 0.005 to 5000 mg of protein, 0.01 to 5000 mg of protein, 0.05 to 5000 mg of protein, 0.05 to 1300 mg of protein, 0.1 to 1300 mg of protein, 0.1 to 500 mg of protein, 0.1 to 100 mg of protein, per liter of wash liquor.

### Nucleic Acid Constructs, Expression & Production of Lipolytic Enzyme Variants

The disclosure also relates to one or more isolated, non-naturally occurring, or recombinant polynucleotide comprising a nucleic acid sequence that encodes one or more variant lipolytic enzyme described herein, or recombinant polypeptide or active fragment thereof. One or more nucleic acid sequence described herein is useful in recombinant production (e.g., expression) of one or more variant lipolytic enzyme described herein, typically through expression of a plasmid expression vector comprising a sequence encoding the one or more variant lipolytic enzyme described herein or fragment thereof. The disclosure provides nucleic acids encoding one or more variant lipolytic enzyme described herein, wherein the variant is a mature form having lipolytic activity. One or more variant lipolytic enzyme described herein is expressed recombinantly with a homologous pro-peptide sequence. Alternatively, one or more variant lipolytic enzyme described herein is expressed recombinantly with a heterologous pro-peptide sequence.

One or more nucleic acid sequence described herein can be generated by using any suitable synthesis, manipulation, and/or isolation techniques, or combinations thereof. For example, one or more polynucleotide described herein may be produced using standard nucleic acid synthesis techniques, such as solid-phase synthesis techniques that are well-known to those skilled in the art. In such techniques, fragments of up to 50 or more nucleotide bases are typically synthesized, then joined (e.g., by enzymatic or chemical ligation methods) to form essentially any desired continuous nucleic acid sequence. The synthesis of the one or more polynucleotide described herein can be also facilitated by any suitable method known in the art. Moreover, customized nucleic acids can be ordered from a variety of commercial sources (e.g., ATUM (DNA 2.0), Newark, CA, USA; Life Tech (GeneArt), Carlsbad, CA, USA; GenScript, Ontario, Canada; Base Clear B. V., Leiden, Netherlands; Integrated DNA Technologies, Skokie, IL, USA; Ginkgo Bioworks (Gen9), Boston, MA, USA; and Twist Bioscience, San Francisco, CA, USA).

Recombinant DNA techniques useful in modification of nucleic acids are well known in the art, such as, e.g., restriction endonuclease digestion, ligation, reverse transcription and cDNA production, and polymerase chain reaction (PCR). One or more polynucleotide described herein may also be obtained by screening cDNA libraries using one or more oligonucleotide probes that can hybridize to or PCR-amplify polynucleotides which encode one or more variant lipolytic enzyme described herein, or recombinant polypeptide or active fragment thereof. Procedures for screening and isolating cDNA clones and PCR amplification procedures are well known to those of skill in the art and described in standard references known to those skilled in the art. One or more polynucleotide described herein can be obtained by altering a naturally occurring polynucleotide backbone (e.g., that encodes one or more variant lipolytic enzyme described herein or reference lipolytic enzyme) by, e.g., a known mutagenesis procedure (e.g., site-directed mutagenesis, site saturation mutagenesis, and in vitro recombination). A variety of methods are known in the art that are suitable for generating modified polynucleotides described herein that encode one or more variant lipolytic enzyme described herein, including, but not limited to, e.g., site-saturation mutagenesis, scanning mutagenesis, insertional mutagenesis, deletion mutagenesis, random mutagenesis, site-directed mutagenesis, and directed-evolution, as well as various other recombinatorial approaches.

The disclosure further is directed to one or more vector comprising one or more variant lipolytic enzyme described herein (e.g., a polynucleotide encoding one or more variant lipolytic enzyme described herein); expression vectors or expression cassettes comprising one or more nucleic acid or polynucleotide sequence described herein; isolated, substantially pure, or recombinant DNA constructs comprising one or more nucleic acid or polynucleotide sequence described herein; isolated or recombinant cells comprising one or more polynucleotide sequence described herein; and compositions comprising one or more such vector, nucleic acid, expression vector, expression cassette, DNA construct, cell, cell culture, or any combination or mixtures thereof.

The disclosure is directed to one or more recombinant cell comprising one or more vector (e.g., expression vector or DNA construct) described herein which comprises one or more nucleic acid or polynucleotide sequence described herein. Some such recombinant cells are transformed or transfected with such at least one vector, although other methods are available and known in the art. Such cells are typically referred to as host cells. Some such cells comprise bacterial cells, including, but not limited to Bacillus sp. cells, such as B. subtilis cells. The disclosure is directed to recombinant cells (e.g., recombinant host cells) comprising one or more variant lipolytic enzyme described herein.

One or more vector described herein is an expression vector or expression cassette comprising one or more polynucleotide sequence described herein operably linked to one or more additional nucleic acid segments required for efficient gene expression (e.g., a promoter operably linked to one or more polynucleotide sequence described herein). A vector may include a transcription terminator and/or a selection gene (e.g., an antibiotic resistant gene) that enables continuous cultural maintenance of plasmid-infected host cells by growth in antimicrobial-containing media. An expression vector may be derived from plasmid or viral DNA, or alternatively, contains elements of both.

For expression and production of a protein of interest (e.g., one or more variant lipolytic enzyme described herein) in a cell, one or more expression vector comprising one or more copy of a polynucleotide encoding one or more variant lipolytic enzyme described herein, and in some instances comprising multiple copies, is transformed into the cell under conditions suitable for expression of the variant. A polynucleotide sequence encoding one or more variant lipolytic enzyme described herein (as well as other sequences included in the vector) is integrated into the genome of the host cell, while alternatively, a plasmid vector comprising a polynucleotide sequence encoding one or more variant lipolytic enzyme described herein remains as autonomous extra-chromosomal element within the cell. The disclosure relates to both extrachromosomal nucleic acid elements as well as incoming nucleotide sequences that are integrated into the host cell genome. The vectors described herein are useful for production of the one or more variant lipolytic enzyme described herein. A polynucleotide construct encoding one or more variant lipolytic enzyme described herein is present on an integrating vector that enables the integration and optionally the amplification of the polynucleotide encoding the variant into the host chromosome. Examples of sites for integration are well known to those skilled in the art. In some embodiments, transcription of a polynucleotide encoding one or more variant lipolytic enzyme described herein is effectuated by a promoter that is the wild-type promoter for the parent enzyme. The promoter might be heterologous to the one or more variant lipolytic enzyme described herein, but is functional in the host cell. Examples of promoters suitable for use in bacterial host cells are well known to those skilled in the art.

One or more variant lipolytic enzyme described herein can be produced in host cells of any suitable microorganism, including bacteria and fungi. One or more variant lipolytic enzyme described herein can be produced in Gram-positive bacteria. The host cells might be Bacillus spp., Streptomyces spp., Escherichia spp., Aspergillus spp., Trichoderma spp., Pseudomonas spp., Corynebacterium spp., Saccharomyces spp., or Pichia spp. One or more variant lipolytic enzyme described herein might be produced by Bacillus sp. host cells. Examples of Bacillus sp. host cells that find use in the production of the one or more variant lipolytic enzyme described herein include, but are not limited to B. licheniformis, B. lentus, B. subtilis, B. amyloliquefaciens, B. brevis, B. stearothermophilus, B. alkalophilus, B. coagulans, B. circulans, B. pumilis, B. thuringiensis, B. clausii, and B. megaterium, as well as other organisms within the genus Bacillus. B. subtilis host cells might be used to produce the variants described herein. U.S. Pat. Nos. 5,264,366 and 4,760,025 describe various Bacillus host strains that can be used to produce one or more variant lipolytic enzyme described herein, although other suitable strains can be used. Examples of suitable host cells are well known to those skilled in the art.

Host cells are transformed with one or more nucleic acid sequence encoding one or more variant lipolytic enzyme described herein using any suitable method known in the art. Methods for introducing a nucleic acid (e.g., DNA) into Bacillus cells or E. coli cells utilizing plasmid DNA constructs or vectors and transforming such plasmid DNA constructs or vectors into such cells are well known. The plasmids might be subsequently isolated from E. coli cells and transformed into Bacillus cells. However, it is not essential to use intervening microorganisms such as E. coli, and hence, a DNA construct or vector might be directly introduced into a Bacillus host. Examples of methods for introducing one or more nucleic acid sequence described herein into host cells are well known to those skilled in the art. Indeed, such methods as transformation, including protoplast transformation and transfection, transduction, and protoplast fusion are well known and suited for use herein.

Alternatively, host cells might be directly transformed with a DNA construct or vector comprising a nucleic acid encoding one or more variant lipolytic enzyme described herein (i.e., an intermediate cell is not used to amplify, or otherwise process, the DNA construct or vector prior to introduction into the host cell). Introduction of a DNA construct or vector described herein into the host cell includes those physical and chemical methods known in the art to introduce a nucleic acid sequence (e.g., DNA sequence) into a host cell without insertion into the host genome. Such methods include, but are not limited to calcium chloride precipitation, electroporation, naked DNA, and liposomes. DNA constructs or vector might be co-transformed with a plasmid, without being inserted into the plasmid, or a selective marker is deleted from the altered Bacillus strain by methods known in the art. The transformed cells are cultured in conventional nutrient media. The suitable specific culture conditions, such as temperature, pH and the like are known to those skilled in the art and are well described in the scientific literature. Such incubation provides a culture (e.g., cell culture) comprising one or more variant lipolytic enzyme or nucleic acid sequence described herein.

Host cells transformed with one or more polynucleotide sequence encoding one or more variant lipolytic enzyme described herein are cultured in a suitable nutrient medium under conditions permitting the expression of the variant, after which the resulting variant is recovered from the culture. The variant produced by the cells is recovered from the culture medium by conventional procedures, including, but not limited to, e.g., separating the host cells from the medium by centrifugation or filtration, precipitating the proteinaceous components of the supernatant or filtrate by means of a salt (e.g., ammonium sulfate), and chromatographic purification (e.g., ion exchange, gel filtration, affinity, etc.).

Alternatively, one or more variant lipolytic enzyme produced by a recombinant host cell is secreted into the culture medium. A nucleic acid sequence that encodes a purification facilitating domain may be used to facilitate purification of the variant. A vector or DNA construct comprising a polynucleotide sequence encoding one or more variant lipolytic enzyme described herein may further comprise a nucleic acid sequence encoding a purification facilitating domain to facilitate purification of the variant. Such methods are well known to those skilled in the art.

A variety of methods can be used to determine the level of production of one or more mature variant lipolytic enzyme described herein in a host cell. Such methods include, but are not limited to, e.g., methods that utilize either polyclonal or monoclonal antibodies specific for the enzyme. Exemplary methods include, but are not limited to, enzyme-linked immunosorbent assays (ELISA), radio immunoassays (RIA), fluorescent immunoassays (FIA), and fluorescent activated cell sorting (FACS). These and other assays are well known in the art. Alternatively, the method that can be used includes the assays provided in Examples 2 and 3.

The disclosure also provides methods for making or producing one or more mature variant lipolytic enzyme described herein. A mature variant does not include a signal peptide or a pro-peptide sequence. Some methods comprise making or producing one or more variant lipolytic enzyme described herein in a recombinant bacterial host cell, e.g., a Bacillus sp. cell (e.g., a B. subtilis cell). The disclosure provides a method of producing one or more variant described herein, wherein the method comprises cultivating a recombinant host cell comprising a recombinant expression vector comprising a nucleic acid sequence encoding one or more variant lipolytic enzyme described herein under conditions conducive to the production of the variant. Some such methods further comprise recovering the variant from the culture.

Further the disclosure provides methods of producing one or more variant lipolytic enzyme described herein, wherein the methods comprise: (a) introducing a recombinant expression vector comprising a nucleic acid encoding the variant into a population of cells (e.g., bacterial cells, such as B. subtilis cells); and (b) culturing the cells in a culture medium under conditions conducive to produce the variant encoded by the expression vector. Some such methods further comprise: (c) isolating the variant from the cells or from the culture medium.

### Compositions

The variant lipolytic enzymes provided herein may be used in the production of various compositions, such as enzyme compositions and cleaning or detergent compositions. Thus, in one embodiment, the present disclosure provides enzyme compositions comprising the variant lipolytic enzymes of the present disclosure, as well as cleaning or detergent compositions comprising the variant lipolytic enzymes provided herein or the enzyme compositions comprising such variant lipolytic enzymes.

As used herein, the “enzyme composition” refers to any enzyme product, preparation or composition, which comprises at least one of the variant lipolytic polypeptides provided herein. Such an enzyme composition may be a spent culture medium or filtrate containing one or more variant lipolytic enzymes, or one or more variant lipolytic enzymes and one or more additional enzymes. Spent culture medium means the culture medium of the host comprising the produced enzymes. Preferably the host cells are separated from the medium after the production. The enzyme composition may be a “whole culture broth” composition, optionally after inactivating the production host(s) or microorganism(s) without any biomass separation, down-stream processing or purification of the desired variant lipolytic enzyme(s), because the variant polypeptides can be secreted into the culture medium, and they display activity in the ambient conditions of the spent culture medium.

The enzyme composition may contain the variant lipolytic enzymes in at least partially purified and isolated form. It may even essentially consist of the desired enzyme or enzymes. If desired, the enzyme compositions may be dried, spray-dried or lyophilized, granulated or the enzymatic activity may be otherwise concentrated and/or stabilized for storage. If required, a desired enzyme may be crystallized or isolated or purified in accordance with conventional methods, such as filtration, extraction, precipitation, chromatography, affinity chromatography, electrophoresis, or the like.

Enzyme granules may be made, for example, by rotary atomization, wet granulation, dry granulation, spray drying, disc granulation, extrusion, pan coating, spheronization, drum granulation, fluid-bed agglomeration, high-shear granulation, fluid-bed spray coating, crystallization, precipitation, emulsion gelation, spinning disc atomization and other casting approaches, and prilling processes. The core of the granule may be the granule itself or the inner nucleus of a layered granule.

In some embodiments, the enzyme compositions comprise a variant lipolytic enzyme as provided herein in combination with one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof. Generally, at least one enzyme coating layer comprises at least one variant lipolytic enzyme.

The enzyme composition can be in any form suitable. For example, the enzyme composition can be in the form of a liquid composition or a solid composition such as solution, dispersion, paste, powder, granule, granulate, coated granulate, tablet, cake, crystal, crystal slurry, gel or pellet.

The enzyme composition can be used in cleaning agents or boosters that are added on top of the detergent during or before the wash and that are for example in the form of liquid, gel, powder, granules or tablets. The enzyme composition and detergent components may also be soaked in a carrier like textiles.

### Cleaning and Fabric Conditioning Compositions

The present invention is directed to cleaning and fabric conditioning compositions (e.g., detergent compositions or laundry detergent compositions or fabric finishers or fabric softeners) comprising at least one variant lipolytic enzyme having polyesterase activity described herein and at least one additional ingredient selected from the group consisting of surfactants, complexing agents (builders) and performance polymers. The compositions generally comprise at least one variant lipolytic enzyme having polyesterase activity described herein and one or more additional detergent components, such as those described herein.

The compositions according to the invention comprise the at least one variant lipolytic enzyme having polyesterase activity at a concentration of in use of 0.001 to 10000 mg/L, or 0.001 to 2000 mg/L, or 0.01 to 5000 mg/L, or 0.01 to 2000 mg/L, or 0.01 to 1300 mg/L, or 0.1 to 5000 mg/L, or 0.1 to 2000 mg/L, or 0.1 to 1300 mg/L, or 1 to 5000 mg/L, or 1 to 1300 mg/L, or 1 to 500 mg/L, or 10 to 5000 mg/L, or 10 to 1300 mg/L, or 10 to 500 mg/L. In another embodiment, the composition may comprise at least one variant lipolytic enzyme in an amount of 0.002 to 5000 mg of protein, such as 0.005 to 1300 mg of protein, or 0.01 to 5000 mg of protein, or 0.01 to 1300 mg of protein, or 0.1 to 5000 mg of protein, or 1 to 1300 mg of protein, preferably 0.1 to 1300 mg of protein, more preferably 1 to 1300 mg of protein, even more preferably 10 to 500 mg of protein, per liter of wash liquor, or in the amount of at least 0.01 ppm active enzyme.

In one embodiment, the composition comprises at least one variant lipolytic enzyme having polyesterase activity described herein, at least one additional detergent component, and optionally one or more additional enzymes, for example at least one cellulase.

The compositions according to the invention may comprise at least one cellulase at a concentration of in use of 0.001 to 10000 mg/L, or 0.001 to 2000 mg/L, or 0.01 to 5000 mg/L, or 0.01 to 2000 mg/L, or 0.01 to 1300 mg/L, or 0.1 to 5000 mg/L, or 0.1 to 2000 mg/L, or 0.1 to 1300 mg/L, or 1 to 5000 mg/L, or 1 to 1300 mg/L, or 1 to 500 mg/L, or 10 to 5000 mg/L, or 10 to 1300 mg/L, or 10 to 500 mg/L. In another embodiment, the composition may comprise at least one cellulase in an amount of 0.002 to 5000 mg of protein, such as 0.005 to 1300 mg of protein, or 0.01 to 5000 mg of protein, or 0.01 to 1300 mg of protein, or 0.1 to 5000 mg of protein, or 1 to 1300 mg of protein, preferably 0.1 to 1300 mg of protein, more preferably 1 to 1300 mg of protein, even more preferably 10 to 500 mg of protein, per liter of wash liquor, or in the amount of at least 0.01 ppm active enzyme.

In some aspects of the invention, the additional detergent component is selected from the group consisting of surfactants, builders, flocculating aid, chelating agents, dye transfer inhibitors, enzymes, enzyme stabilizers, enzyme inhibitors, catalytic materials, bleaching agents, bleach activators, bleach catalysts, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric hueing agents, anti-foaming agents, dispersants, processing aids, pH control agents, alkalinity sources, solubilizing agents photoactivators, fluorescers, fabric conditioners, hydrolysable surfactants, preservatives, anti-oxidants, anti-shrinkage agents, anti-wrinkle agents, germicides, fungicides, color speckles, anti-corrosion agents, silver care, anti-tarnish, filler salts, colorants, soil release polymers and/or pigments. In various embodiments, the at least one additional ingredient is selected from surfactants, complexing agents (builders) and performance polymers or combinations thereof.

In particular, a combination of a cleaning composition according to the invention with one or more further ingredients of the composition is advantageous, since, in preferred embodiments according to the invention, such a cleaning composition has improved cleaning performance by virtue of resulting synergisms. In particular, combining a cleaning composition according to the invention with a surfactant and/or a builder and/or a performance polymer and/or a peroxygen compound and/or a bleach activator can result in such a synergism. Particularly, the combination of the inventive enzyme combination (lipolytic enzyme having polyesterase activity described herein and optionally cellulase) with performance polymer, e.g., soil release polymers, anti-redeposition agents or dye transfer inhibitors, leads to synergistic effects regarding improvement of anti-pilling and/or anti-graying performance and/or moisture management.

Advantageous ingredients of cleaning compositions according to the invention are disclosed in international patent application WO 2009/121725, starting at the penultimate paragraph of page 5 and ending after the second paragraph on page 13. Reference is expressly made to this disclosure and the disclosure therein is incorporated in the present patent application by reference.

Enzyme component weights are based on total active protein. All percentages and ratios are calculated by weight unless otherwise indicated. All percentages and ratios are calculated based on the total composition unless otherwise indicated. In laundry detergent compositions, the enzyme levels are expressed in ppm, which equals mg active protein/kg detergent composition.

### Surfactants

The cleaning compositions according to the invention might comprise at least one compound from the class of surfactants, in particular selected from anionic, non-ionic, cationic, zwitterionic or amphoteric surfactants, or any mixture thereof. In some embodiments, the compositions comprise from 0.1 to 60 wt. %, or from 1 to 50 wt. %, or from 5 to 40 wt. % surfactant relative to the total weight of the composition.

When included therein the cleaning composition according to the invention will usually comprise from 1 to 40 wt. % of an anionic surfactant, e.g., from 5 to 30 wt. %, in particular from 5 to 15 wt. %, or from 15 to 20 wt. %, or from 20 to 25 wt. %, preferably 2 to 6 wt. %, more preferably 3 to 5 wt. %.

Suitable surfactants are, e.g., anionic surfactants of the formula (I)

R—SO₃⁻Y⁺ (I),

in which R represents a linear or branched, unsubstituted alkyl aryl functional group, and Y⁺ represents a monovalent cation or the n^{th} part of an n-valent cation, the alkali metal ions, including Na⁺ or K⁺, being preferred in this case, with Na⁺ being most preferred. Further cations Y⁺ can be selected from NH₄⁺, ½ Zn²⁺, ½ Mg²⁺, ½ Ca²⁺, ½ Mn²⁺, and mixtures thereof.

“Alkyl aryl,” as used herein, refers to organic functional groups that consist of an alkyl functional group and an aromatic functional group. Typical examples of functional groups of this kind include, but are not restricted to, alkyl benzene functional groups, such as benzyl, butyl benzene functional groups, nonyl benzene functional groups, decyl benzene functional groups, undecyl benzene functional groups, dodecyl benzene functional groups, tridecyl benzene functional groups and the like.

Such surfactants might be selected from linear or branched alkyl benzene sulfonates of the formula (A-1):

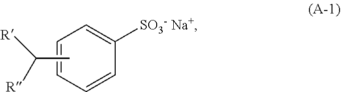

in which R′ and R″ together comprise 9 to 19, preferably 11 to 15, in particular 11 to 13, C atoms.

A very particularly preferred surfactant can be described by formula (A-1a):

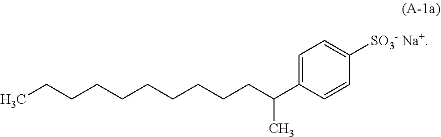

In various embodiments, the compound of the formula (I) is preferably the sodium salt of a linear alkyl benzene sulfonate.

In various embodiments, the cleaning compositions according to the invention might comprise at least one anionic surfactant of the formula (II):

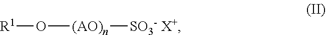

in which R¹ represents a linear or branched, substituted or unsubstituted alkyl, aryl or alkyl aryl functional group, preferably a linear, unsubstituted alkyl functional group, particularly preferably a fatty alcohol functional group. Preferred functional groups R¹ are selected from decyl, undecyl, dodecyl, tridecyl, tetradecyl, pentadecyl, hexadecyl, heptadecyl, octadecyl, nonadecyl, eicosyl functional groups and mixtures thereof, the representatives having an even number of C atoms being preferred. Particularly preferred functional groups R¹ are derived from C₁₂₋₁₈ fatty alcohols, e.g., from coconut fatty alcohol, tallow fatty alcohol, lauryl, myristyl, cetyl or stearyl alcohol or from C₁₀₋₂₀ oxo alcohols.

AO represents an ethylene oxide (EO) group or propylene oxide (PO) group, preferably an ethylene oxide group. The index n represents an integer of from 1 to 50, preferably from 1 to 20, and in particular from 2 to 10. Very particularly preferably, n represents the numbers 2, 3, 4, 5, 6, 7 or 8. X⁺ represents a monovalent cation or the n^{th} part of an n-valent cation, in this case the alkali metal ions, which include Na⁺ or K⁺, being preferred, Na⁺ being most preferred. Further cations X⁺ can be selected from NH₄+, ½ Zn²⁺, ½ Mg²⁺, ½ Ca²⁺, ½ Mn²⁺ and mixtures thereof.

Cleaning compositions according to the invention might comprise at least one anionic surfactant selected from fatty alcohol ether sulfates of the formula (A 2):

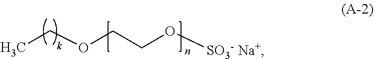

where k=11 to 19, and n=2, 3, 4, 5, 6, 7 or 8. Particularly preferred representatives are Na—C₁₂₋₁₄ fatty alcohol ether sulfates having 2 EO (k=11 to 13, n=2 in formula (A-2)).

Other anionic surfactants that can be used are the alkyl sulfates of the formula (III):

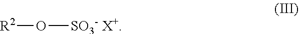

In this formula (III), R² represents a linear or branched, substituted or unsubstituted alkyl functional group, preferably a linear, unsubstituted alkyl functional group, particularly preferably a fatty alcohol functional group. Preferred functional groups R² are selected from decyl, undecyl, dodecyl, tridecyl, tetradecyl, pentadecyl, hexadecyl, heptadecyl, octadecyl, nonadecyl, eicosyl functional groups and mixtures thereof, the representatives having an even number of C atoms being preferred.

Particularly preferred functional groups R² are derived from C₁₂₋₁₈ fatty alcohols, e.g., from coconut fatty alcohol, tallow fatty alcohol, lauryl, myristyl, cetyl or stearyl alcohol or from C₁₀₋₂₀ oxo alcohols. X⁺ represents a monovalent cation or the n^{th} part of an n-valent cation, in this case the alkali metal ions, which include Na⁺ or K⁺, being preferred, Na⁺ being most preferred. Further cations X⁺ can be selected from NH₄+, ½ Zn²⁺, ½ Mg²⁺, ½ Ca²⁺, ½ Mn²⁺, and mixtures thereof.

Further surfactants might be selected from fatty alcohol sulfates of the formula (A-3):

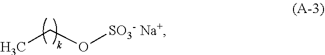

where k=11 to 19. Very particularly preferred representatives are Na—C₁₂₋₁₄ fatty alcohol sulfates (k=11 to 13 in formula (A-3)).

In various embodiments, the cleaning composition according to the invention might comprise, in addition to the anionic surfactants described above, in particular those of the formulas (I) to (III), or alternatively at least one other surfactant. Other alternative or additional surfactants are, in particular, further anionic surfactants, non-ionic surfactants and mixtures thereof, but also cationic, zwitterionic and amphoteric surfactants.

Anionic surfactants include but are not limited to linear alkyl benzene sulfonates (LAS), isomers of LAS, branched alkyl benzene sulfonates (BABS), phenyl alkane sulfonates, α-olefin sulfonates (AOS), olefin sulfonates, alkene sulfonates, alkane-2,3-diylbis-(sulfates), hydroxy alkane sulfonates and disulfonates, alkyl sulfates (AS) such as sodium dodecyl sulfate (SDS), fatty alcohol sulfates (FAS), primary alcohol sulfates (PAS), alcohol ether sulfates (AES or AEOS or FES, also known as alcohol ethoxy sulfates or fatty alcohol ether sulfates), secondary alkane sulfonates (SAS), paraffin sulfonates (PS), ester sulfonates, sulfonated fatty acid glycerol esters, α-sulfo fatty acid methyl esters (α-SFMe or SES) including methyl ester sulfonate (MES), alkyl- or alkenyl succinic acid, dodecenyl/tetradecenyl succinic acid (DTSA), fatty acid derivatives of amino acids, diesters and monoesters of sulfo succinic acid or salt of fatty acids (soap), and combinations thereof. When included therein the cleaning composition according to the invention will usually comprise from 0.2 to 40 wt. % of a non-ionic surfactant, e.g., from 0.5 to 30 wt. %, in particular from 1 to 20 wt. %, from 3 to 10 wt. %, or from 3 to 5 wt. %, from 8 to 12 wt. %, or from 10 to 12 wt. %.

In various embodiments, the cleaning composition according to the invention might comprise at least one non-ionic surfactant, in particular at least one fatty alcohol alkoxylate.

Suitable non-ionic surfactants are those of the formula (IV):

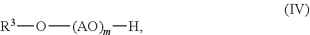

in which R³ represents a linear or branched, substituted or unsubstituted alkyl functional group, preferably a linear, unsubstituted alkyl functional group, particularly preferably a fatty alcohol functional group. Preferred functional groups R³ are selected from decyl, undecyl, dodecyl, tridecyl, tetradecyl, pentadecyl, hexadecyl, heptadecyl, octadecyl, nonadecyl, eicosyl functional groups and mixtures thereof, the representatives having an even number of C atoms being preferred. Particularly preferred functional groups R³ are derived from C₁₂₋₁₈ fatty alcohols, e.g., from coconut fatty alcohol, tallow fatty alcohol, lauryl, myristyl, cetyl or stearyl alcohol or from C₁₀₋₂₀ oxo alcohols.

AO represents an ethylene oxide (EO) group or propylene oxide (PO) group, preferably an ethylene oxide group. The index m represents an integer from 1 to 50, preferably from 1 to 20, and in particular from 2 to 10. Very particularly preferably, m represents the numbers 2, 3, 4, 5, 6, 7 or 8.

The fatty alcohol alkoxylates might be compounds of the formula (V):

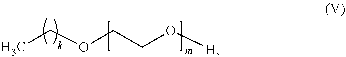

where k=11 to 19, and m=2, 3, 4, 5, 6, 7 or 8. Very particularly preferred representatives are C₁₂₋₁₈ fatty alcohols having 7 EO (k=11 to 17, m=7 in formula (V)).

Further non-ionic surfactants which might be comprised in the compositions according to the invention include, but are not limited to, alkyl glycosides, alkoxylated alkyl fatty acid esters, amine oxides, fatty acid alkanol amides, hydroxy mixed ethers, sorbitan fatty acid esters, polyhydroxy fatty acid amides and alkoxylated alcohols.

Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkyl phenol ethoxylates (APE), nonyl phenol ethoxylates (NPE), alkyl polyglycosides (APG), alkoxylated amines, fatty acid monoethanol amides (FAM), fatty acid diethanol amides (FADA), ethoxylated fatty acid monoethanol amides (EFAM), propoxylated fatty acid monoethanol amides (PFAM), polyhydroxy alkyl fatty acid amides, or N-acyl N-alkyl derivatives of glucosamine (glucamides, GA, or fatty acid glucamides, FAGA), as well as products available under the trade names SPAN and TWEEN, and combinations thereof. Commercially available non-ionic surfactants include Plurafac™, Lutensol™ and Pluronic™ range from BASF, Dehypon™ series from Cognis and Genapol™ series from Clariant.

When included therein the cleaning composition according to the invention will usually comprise from 1 to 40 wt. % of a cationic surfactant, e.g., from 0.5 to 30 wt. %, in particular from 1 to 20 wt. %, or from 3 to 10 wt. %, or from 3 to 5 wt. %, from 8 to 12 wt. % or from 10 to 12 wt. %.

Suitable cationic surfactants are, inter alia, the quaternary ammonium compounds of the formula (R^{vi})(R^{vii})(R^{viii})(R^{ix})N⁺ X⁻, in which R^{vi} to R^{ix} denote four identical or different, and in particular two long-chain and two short-chain, alkyl functional groups, and X⁻ denotes an anion, in particular a halide ion, e.g., didecyl dimethyl ammonium chloride, alkyl benzyl didecyl ammonium chloride and mixtures thereof. Further suitable cationic surfactants are the quaternary surface-active compounds, in particular having a sulfonium, phosphonium, iodonium or arsonium group, which are also known as antimicrobial washing agents. By using quaternary surface-active compounds having an antimicrobial action, the composition can be designed having an antimicrobial effect or whose antimicrobial effect, which may already be present due to other ingredients, can be improved.

Non-limiting examples of cationic surfactants include alkyl dimethyl ethanol amine quat (ADMEAQ), cetyl trimethyl ammonium bromide (CTAB), dimethyl distearyl ammonium chloride (DSDMAC), and alkyl benzyl dimethyl ammonium, alkyl quaternary ammonium compounds, alkoxylated quaternary ammonium (AQA) compounds, ester quats, and combinations thereof.

When included therein the cleaning composition according to the invention will usually comprise from 0.01 to 10 wt. % of a semipolar (amphoteric) or zwitterionic surfactant. Suitable amphoteric surfactants are, e.g., betaines, such as alkyl dimethyl betaines, in particular those of the formula (Rⁱⁱⁱ)(R^{iv})(R^{v})N⁺CH₂COO⁻, in which Rⁱⁱⁱ denotes an alkyl functional group, which is optionally interrupted by heteroatoms or heteroatom groups, having 8 to 25, preferably 10 to 21, carbon atoms, and R^{iv} and R^{v} denote identical or different alkyl functional groups having 1 to 3 carbon atoms, in particular C₁₀₋₁₈ alkyl dimethyl carboxy methyl betaine and C₁₁₋₁₇ alkyl amido propyl dimethyl carboxy methyl betaine. Further non-limiting examples of zwitterionic surfactants include sulfo betaines and amine oxides (AO), such as alkyl dimethyl amine oxide, N-(coco alkyl)-N,N-dimethyl amine oxide and N-(tallow-alkyl)-N,N-bis-(2-hydroxy ethyl)-amine oxide, and combinations thereof.

In various embodiments, the total amount of surfactants based on the weight of the agent is 2 to 50 wt. %, preferably 4 to 35 wt. %, more preferably 5 to 30 or 5 to 25 wt. %, even more preferably 10 to 20 wt. %, even more preferably 14 to 20 wt. %, most preferably 14 to 18 wt. %, the (linear) alkyl benzene sulfonates, if present, being present at most in an amount of from 0.001 to 30 wt. %, preferably 0.001 to 10 wt. %, more preferably 2 to 6 wt. %, even more preferably 3 to 5 wt. %, relative to the total weight of the composition.

In a further embodiment, the cleaning compositions according to the invention comprise a surfactant mixture that includes, but is not limited to 5 to 15% anionic surfactants, <5% non-ionic surfactants, cationic surfactants, phosphonates, soap, enzymes, perfume, butyl phenyl methyl propionate, geraniol, zeolite, polycarboxylates, hexyl cinnamal, limonene, cationic surfactants, citronellol, and benzisothiazolinone.

### Builders and Co-Builders

The cleaning compositions according to the invention might comprise at least one water-soluble and/or water-insoluble, organic and/or inorganic builder. These builders/co-builders are also referred to as “complexing agents” or “complex builders” herein.

The builders that can generally be used include, in particular, the amino carboxylic acids and their salts, zeolites, silicates, carbonates, organic (co)builders and—where there are no ecological prejudices against their use—also the phosphates. However, the cleaning compositions according to the invention are preferably phosphate-free.

The water-soluble organic builders include polycarboxylic acids, in particular citric acid and saccharic acids, monomeric and polymeric amino polycarboxylic acids, in particular methyl glycine diacetic acid (MGDA), nitrile triacetic acid, ethylene diamine tetraacetic acid (EDTA) and polyaspartic acid, polyphosphonic acids, in particular amino tris-(methylene phosphonic acid), ethylene diamine tetrakis-(methylene phosphonic acid), diethylene triamine penta-(methylene phosphonic acid) (DTPMP) and 1-hydroxy ethane-1,1-diphosphonic acid (HEDP), polymeric hydroxy compounds such as dextrin, and polymeric (poly)carboxylic acids, polymeric acrylic acids, methacrylic acids, maleic acids, and mixed polymers thereof, which may also comprise, in the polymer, small portions of polymerizable substances, without a carboxylic acid functionality. Compounds of this class which are suitable, although less preferred, are copolymers of acrylic acid or methacrylic acid with vinyl ethers, such as vinyl methyl ethers, vinyl esters, ethylene, propylene, and styrene, in which the proportion of the acid is at least 50 wt. %. The organic builders may, in particular for the production of liquid compositions, be used in the form of aqueous solutions, preferably in the form of 30 to 50 wt. % aqueous solutions. All mentioned acids are generally used in the form of their water-soluble salts, in particular their alkali salts. In various embodiments, the builders/co-builders comprise citric acid/citrate, MGDA, EDTA, or a combination thereof. In various embodiments, the compositions comprise EDTA and citrate/citric acid.

Organic builders, if desired, can be comprised in amounts of up to 40 wt. %, in particular up to 25 wt. %, and preferably from 1 to 8 wt. %. Amounts close to the stated upper limit are preferably used in paste-form or liquid, in particular water-containing, compositions according to the invention. Laundry post-treatment compositions according to the invention, such as softeners, can optionally also be free of organic builders.

Suitable water-soluble inorganic builder materials are, in particular, alkali silicates and, if there are no concerns about their use, also polyphosphates, preferably sodium triphosphate. In particular crystalline or amorphous alkali aluminosilicates, if desired, can be used as water-insoluble, water-dispersible inorganic builder materials in amounts of up to 50 wt. %, preferably no greater than 40 wt. %, and in liquid agents in particular in amounts of from 1 to 5 wt. %. Among these, crystalline sodium aluminosilicates of washing agent quality, in particular zeolite A, P and optionally X, are preferred. Amounts close to the stated upper limit are preferably used in solid particulate agents. Suitable aluminosilicates have in particular no particles having a particle size greater than 30 μm and preferably comprise at least 80 wt. % of particles having a size smaller than 10 μm.

Suitable substitutes or partial substitutes for the stated aluminosilicate are crystalline alkali silicates, which may be present alone or in a mixture with amorphous silicates. The alkali silicates that can be used in the compositions according to the invention as builders preferably have a molar ratio of alkali oxide to SiO₂ of less than 0.95, in particular from 1:1.1 to 1:12, and may be present in amorphous or crystalline form. Preferred alkali silicates are sodium silicates, in particular amorphous sodium silicates having a Na₂O:SiO₂ molar ratio of from 1:2 to 1:2.8. Preferably used as crystalline silicates, which may be present alone or in a mixture with amorphous silicates, are crystalline phyllosilicates of general formula Na₂SiₓO₂ₓ₊₁·y H₂O, where x, referred to as the module, is a number from 1.9 to 4, y is a number from 0 to 20, and preferred values for x are 2, 3 or 4. Preferred crystalline phyllosilicates are those in which x in the stated general formula assumes the values 2 or 3. In particular, both beta-sodium and delta-sodium disilicates (Na₂Si₂O₅·y H₂O) are preferred. Practically water-free crystalline alkali silicates of the above general formula, in which x is a number from 1.9 to 2.1, which alkali silicates are produced from amorphous alkali silicates, may also be used in compositions according to the invention. In a further preferred embodiment of compositions according to the invention, a crystalline sodium phyllosilicate having a module of from 2 to 3, as can be produced from sand and soda, is used. Crystalline sodium silicates having a module in the range of from 1.9 to 3.5 are used in a further preferred embodiment of compositions according to the invention. If alkali aluminosilicate, in particular zeolite, is also present as an additional builder, the weight ratio of aluminosilicate to silicate, in each case based on water-free active substances, is preferably from 1:10 to 10:1. In compositions comprising both amorphous and crystalline alkali silicates, the weight ratio of amorphous alkali silicate to crystalline alkali silicate is preferably from 1:2 to 2:1 and in particular from 1:1 to 2:1. Builders are, if desired, preferably comprised in the compositions according to the invention in amounts of up to 60 wt. %, in particular from 5 to 40 wt. %. Water-soluble builders are particularly preferred in liquid formulations. Laundry post-treatment compositions according to the invention, for example softeners, are preferably free of inorganic builders.

### Additional Enzymes

The cleaning compositions according to the invention might comprise further enzymes in addition to the polyesterase. Alternatively, they may also comprise other hydrolytic enzymes or other enzymes in a concentration that is expedient for the effectiveness of the cleaning composition. One embodiment of the invention thus represents cleaning compositions which comprise one or more enzymes. All enzymes which can develop catalytic activity in cleaning compositions according to the invention, in particular acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, DNases, endoglucanases, endo-β-1,4-glucanases, endo-beta-mannanases, esterases, exo-mannanases, galactanases, glucoamylases, haloperoxygenases, hemicellulases, hexoaminidases, hyaluronidases, keratinases, laccases, lactases, ligninases, lipases, lipoxygenases, mannanases, metalloproteases, nucleases (e.g., deoxyribonucleases and ribonucleases), oxidases, oxidoreductases, pectate lyases, pectin acetyl esterases, pectinases, pectin lyases, pentosanases, peroxidases, phenoloxidases, phosphatases, phospholipases, phytases, polygalacturonases, polyesterases, proteases, pullulanases, reductases, rhamnogalacturonases, β-glucanases, tannases, transglutaminases, xanthanases, xylan acetyl esterases, xylanases, xyloglucanases, xylosidases, and any combination or mixture thereof, can preferably be used as such additional enzymes. Some embodiments are directed to a combination of enzymes (i.e., a “cocktail”) comprising enzymes like amylase, protease, cellulase, lipase, mannanase, and/or nuclease in conjunction with one or more variant lipolytic enzyme in the compositions provided herein. Specific enzymes suitable for the detergent compositions of the invention are described below.

Enzymes are included in the composition advantageously in an amount of from 1×10⁻⁸ to 5 wt. % active enzyme protein. Increasingly preferably, each enzyme is included in compositions according to the invention in an amount of from 1×10⁻⁷ to 3 wt. %, from 0.00001 to 1 wt. %, from 0.00005 to 0.5 wt. %, from 0.0001 to 0.1 wt. % and particularly preferably from 0.0001 to 0.05 wt. %, active enzyme protein. Particularly preferably, the enzymes exhibit synergistic cleaning performance on specific stains or spots, i.e., the enzymes comprised in the agent composition support one another in their cleaning performance. Synergistic effects can arise not only between different enzymes, but also between one or more enzymes and other ingredients of the composition according to the invention.

The protein concentration can be determined using known methods, e.g., the BCA method (bicinchoninic acid; 2,2′-bichinolyl-4,4′-dicarboxylic acid) or the Biuret method. The active protein concentration is determined by titrating the active centers using a suitable irreversible inhibitor (e.g., phenyl methyl sulfonyl fluoride (PMSF) for proteases) and determining the residual activity (M. Bender et al., J. Am. Chem. Soc. 88 (24): 5890-5913, 1966).

In some embodiments, cleaning compositions according to the invention comprise at least one variant lipolytic enzyme in combination with at least one protease. In one embodiment, the composition comprises from about 0.00001 to 5 wt. %, about 0.0001 to 3 wt. %, about 0.001 to 2 wt. %, about 0.001 to 1 wt. %, or about 0.005 to 0.5 wt. % protease (active enzyme protein) by weight composition. The protease for use in combination with the at least one variant lipolytic enzyme in the compositions of the present invention include any polypeptide having protease activity. In one embodiment, the additional protease is a serine protease. In another embodiment, the additional protease is a metalloprotease, a fungal subtilisin, or an alkaline microbial protease or a trypsin-like protease. Suitable proteases include those of animal, vegetable or microbial origin. In some embodiments, the protease is a microbial protease. In other embodiments, the protease is a chemically or genetically modified mutant. In another embodiment, the protease is subtilisin like protease or a trypsin-like protease. In other embodiments, the additional protease does not comprise cross-reactive epitopes with the variant as measured by antibody binding or other assays available in the art. Exemplary subtilisin proteases include those derived from, e.g., Bacillus (e.g., BPN′, Carlsberg, subtilisin 309, subtilisin 147, and subtilisin 168), or fungal origin. Exemplary additional proteases include, but are not limited to trypsin (e.g., of porcine or bovine origin) and the Fusarium protease. Exemplary commercial proteases include, but are not limited to MAXATASE®, MAXACAL™, MAXAPEM™ OPTICLEAN®, OPTIMASE®, PROPERASE®, PURAFECT®, PURAFECT® OXP, PURAMAX™, EXCELLASE™, PREFERENZ™ proteases (e.g., P100, P110, P280), EFFECTENZ™ proteases (e.g., P1000, P1050, P2000), EXCELLENZ™ proteases (e.g., P1000), ULTIMASE®, and PURAFAST™ (DuPont); ALCALASE®, BLAZE®, BLAZE® variants, BLAZE® EVITY®, BLAZE® EVITY® 16L, CORONASE®, SAVINASE®, SAVINASE® ULTRA, SAVINASE® EVITY®, SAVINASE® EVERIS®, PRIMASE®, DURAZYM™, POLARZYME®, OVOZYME®, KANNASE®, LIQUANASE®, LIQUANASE EVERIS®, NEUTRASE®, PROGRESS UNO®, RELASE®, and ESPERASE® (Novozymes); BLAP™ and BLAP™ variants (Henkel); LAVERGY™ PRO 104 L, LAVERGY™ PRO 106 LS, LAVERGY™ PRO 114 LS (BASF), KAP (B. alkalophilus subtilisin (Kao)) and BIOTOUCH® (AB Enzymes).

In some embodiments, cleaning compositions according to the invention comprise at least one variant lipolytic enzyme in combination with one or more amylases. In one embodiment, the composition comprises from about 0.00001 to 5 wt. %, about 0.0001 to 3 wt. %, about 0.001 to 2 wt. %, about 0.001 to 1 wt. %, or about 0.005 to 0.5 wt. % amylase (active enzyme protein) by weight composition. Any amylase (e.g., alpha and/or beta) suitable for use in alkaline solutions may be useful to include in such composition. An exemplary amylase can be a chemically or genetically modified mutant. Exemplary commercial amylases include, but are not limited to AMPLIFY®, AMPLIFY® PRIME, DURAMYL®, TERMAMYL®, FUNGAMYL®, STAINZYME®, STAINZYME PLUS®, STAINZYME ULTRA® EVITY®, and BAN™ (Novozymes); EFFECTENZ™ S1000, POWERASE™, PREFERENZ™ (S100, S110, S210), EXCELLENZ™ (S2000, S3300), RAPIDASE® and MAXAMYL® P (DuPont).

In some embodiments, cleaning compositions according to the invention comprise at least one variant lipolytic enzyme in combination with one or more additional lipases. In some embodiments, the composition comprises from about 0.00001 to 5 wt. %, about 0.0001 to 3 wt. %, about 0.001 to 2 wt. %, about 0.001 to 1 wt. %, or about 0.005 to 0.5 wt. % lipase (active enzyme protein) by weight composition. An exemplary lipase can be a chemically or genetically modified mutant. Exemplary lipases include, but are not limited to, e.g., those of bacterial or fungal origin, such as, e.g., H. lanuginosa lipase, T. lanuginosa lipase, Rhizomucor miehei lipase, Candida lipase, such as C. antarctica lipase (e.g., C. antarctica lipase A or B), Pseudomonas lipases such as P. alcaligenes and P. pseudoalcaligenes lipase, P. cepacia lipase, P. stutzeri lipase, P. fluorescens lipase, Bacillus lipase B. stearothermophilus lipase, and B. pumilus lipase. Exemplary cloned lipases include, but are not limited to Penicillium camembertii lipase, Geotrichum candidum lipase, and various Rhizopus lipases, such as, R. delemar lipase, R. niveus lipase and R. oryzae lipase. Other lipolytic enzymes, such as cutinases, may also find use in one or more composition according to the invention, including, but not limited to, e.g., cutinase derived from Pseudomonas mendocina and/or Fusarium solani pisi. Exemplary commercial lipases include, but are not limited to M1 LIPASE™, LUMA FAST™, and LIPOMAX™ (DuPont); LIPEX®, LIPEX® EVITY, LIPOCLEAN®, LIPOLASE® and LIPOLASE® ULTRA (Novozymes); and LIPASE P™ (Amano Pharmaceutical Co. Ltd).

In some embodiments, cleaning compositions according to the invention comprise at least one variant lipolytic enzyme in combination with one or more mannanases. In one embodiment, the composition comprises from about 0.00001 to 5 wt. %, about 0.0001 to 3 wt. %, about 0.001 to 2 wt. %, about 0.001 to 1 wt. %, or about 0.005 to 0.5 wt. % mannanase (active enzyme protein) by weight composition. Any suitable mannanase may find use in a composition according to the invention. An exemplary mannanase can be a chemically or genetically modified mutant. Exemplary commercial mannanases include, but are not limited to MANNAWAY® (Novozymes) and EFFECTENZ™ M1000, EFFECTENZ™ M2000, PREFERENZ® M100, MANNASTAR®, and PURABRITE™ (DuPont).

In some embodiments, cleaning compositions according to the invention comprise at least one variant lipolytic enzyme in combination with one or more cellulases. In one embodiment, the composition comprises from about 0.00001 to 5 wt. %, about 0.0001 to 3 wt. %, about 0.001 to 2 wt. %, about 0.001 to 1 wt. %, or about 0.005 to 0.5 wt. % cellulase (active enzyme protein) by weight composition. Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259. Especially suitable cellulases are the alkaline or neutral cellulases having color care benefits. Examples of such cellulases are cellulases described in EP 0495257, EP 0531372, WO 96/11262, WO 96/29397, WO 98/08940. Other examples are cellulase variants such as those described in WO 94/07998, EP 0531315, U.S. Pat. Nos. 5,457,046, 5,686,593, 5,763,254, WO 95/24471, WO 98/12307 and WO 99/01544. Examples of cellulases exhibiting endo-β-1,4-glucanase activity (EC 3.2.1.4) are described in WO 2002/099091, e.g., those having a sequence of at least 97% identity to the amino acid sequence of positions 1 to 773 of SEQ ID NO:2 of WO 2002/099091. Further example may include a GH44 xyloglucanase, e.g., a xyloglucanase enzyme having a sequence of at least 60% identity to positions 40 to 559 of SEQ ID NO:2 of WO 01/62903. Other examples of cellulases include the GH45 cellulases described in WO 96/29397, and especially variants thereof having substitution, insertion and/or deletion at one or more of the positions corresponding to the following positions in SEQ ID NO:8 of WO 2002/099091: 2, 4, 7, 8, 10, 13, 15, 19, 20, 21, 25, 26, 29, 32, 33, 34, 35, 37, 40, 42, 42a, 43, 44, 48, 53, 54, 55, 58, 59, 63, 64, 65, 66, 67, 70, 72, 76, 79, 80, 82, 84, 86, 88, 90, 91, 93, 95, 95d, 95h, 95j, 97, 100, 101, 102, 103, 113, 114, 117, 119, 121, 133, 136, 137, 138, 139, 140a, 141, 143a, 145, 146, 147, 150e, 150j, 151, 152, 153, 154, 155, 156, 157, 158, 159, 160c, 160e, 160k, 161, 162, 164, 165, 168, 170, 171, 172, 173, 175, 176, 178, 181, 183, 184, 185, 186, 188, 191, 192, 195, 196, 200, and/or 20, preferably selected among P19A, G20K, Q44K, N48E, Q119H or Q146R. Commercially available cellulases include Celluzyme™, Carezyme™, Carezyme Premium™, Celluclean™, Celluclean Classic™, Cellusoft™, Endolase®, Renozyme® and Whitezyme™ (Novozymes A/S), Clazinase™ and Puradax HAM (Genencor International Inc.), KAC-500(B)™ (Kao Corporation), Revitalenz™ 1000, Revitalenz™ 2000 and Revitalenz™ 3000 (DuPont), and Ecostone® and Biotouch® series (AB Enzymes).

In some embodiments, the cleaning compositions according to the invention comprise one or more enzyme stabilizer. In some embodiments, the enzyme stabilizer is a water-soluble source of calcium and/or magnesium ions. In some embodiments, the enzyme stabilizers include oligosaccharides, polysaccharides, and inorganic divalent metal salts, including alkaline earth metals, such as calcium salts. In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), tin (II), cobalt (II), copper (II), nickel (II), and oxovanadium (IV)). Chlorides and sulfates also find use in some embodiments. Exemplary oligosaccharides and polysaccharides (e.g., dextrins) are described, e.g., in WO 2007/145964. In some embodiments, the compositions according to the invention comprise reversible protease inhibitors selected from a boron-containing compound (e.g., boric acid, borate, 4-formyl phenyl boronic acid, and phenyl-boronic acid derivatives, such as, e.g., described in WO 96/41859); a peptide aldehyde (such as, e.g., described in WO 2009/118375 and WO 2013/004636), and combinations thereof.

In the cleaning compositions according to the invention, the enzymes to be used may furthermore be formulated together with accompanying substances, e.g., from fermentation. In liquid formulations, the enzymes are preferably used as enzyme liquid formulations.

The enzymes are generally not provided in the form of pure protein, but rather in the form of stabilized, storable and transportable preparations. These pre-formulated preparations include, e.g., the solid preparations obtained through granulation, extrusion, or lyophilization or, in particular in the case of liquid or gel agents, solutions of the enzymes, advantageously maximally concentrated, low-water, and/or supplemented with stabilizers or other auxiliaries.

Alternatively, the enzymes can also be encapsulated, for both the solid and the liquid administration form, e.g., by spray-drying or extrusion of the enzyme solution together with a preferably natural polymer or in the form of capsules, e.g., those in which the enzymes are enclosed in a set gel, or in those of the core-shell type, in which an enzyme-containing core is coated with a water-, air-, and/or chemical-impermeable protective layer. Other active ingredients such as stabilizers, emulsifiers, pigments, bleaching agents, or dyes can additionally be applied in overlaid layers. Such capsules are applied using inherently known methods, e.g., by shaking or roll granulation or in fluidized bed processes. Such granules are advantageously low in dust, e.g., due to the application of polymeric film-formers, and stable in storage due to the coating.

Moreover, it is possible to formulate two or more enzymes together, such that a single granule exhibits a plurality of enzyme activities.

Reducing agents and antioxidants increase the stability of the enzymes against oxidative decay; for this purpose, sulfur-containing reducing agents are common, such as sodium sulfite and reducing sugars.

### Solvents

In one embodiment, the cleaning compositions according to the invention are liquid and comprise water as the main solvent, i.e., they are aqueous agents. The water content of the aqueous cleaning composition according to the invention is usually 15 to 70 wt. %, preferably 20 to 60 wt. %. In various embodiments, the water content is more than 5 wt. %, preferably more than 15 wt. % and particularly preferably more than 50 wt. %, of water, in each case based on the total weight of composition.

In addition, non-aqueous solvents can be added to the composition. Suitable non-aqueous solvents include monovalent or polyvalent alcohols, alkanol amines or glycol ethers, if they can be mixed with water in the stated concentration range. Preferably, the solvents are selected from ethanol, n-propanol, i-propanol, butanol, glycol, propane diol, butane diol, methyl propane diol, glycerol, diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol mono methyl ether, dipropylene glycol mono ethyl ether, methoxy triglycol, ethoxy triglycol, butoxy triglycol, 1-butoxy ethoxy-2-propanol, 3-methyl-3-methoxy butanol, propylene-glycol-t-butyl ether, di-n-octyl ether and mixtures thereof.

The one or more non-aqueous solvents are usually present in an amount of from 0.1 to 10 wt. %, preferably 1 to 8 wt. %, more preferably 0.1 to 5 wt. %, based on the total composition.

### Performance Polymers

Performance polymers include various components, namely polymers, which impart an additional stain removal effect and/or textile care effect to the detergent. These include, e.g., soil release polymers, anti-redeposition agents, dispersants, dye transfer inhibitors and graying inhibitors. When incorporated into compositions according to the invention performance polymers are included in amounts of 0.05 to 5 wt.-%, preferably 0.1 to 2 wt. %, in particular 0.05 to 0.5 wt. %, in relation to the total weight of the composition. The compositions according to the invention may also comprise components which positively influence the oil and grease washability from textiles, so-called soil release agents. This effect is particularly evident when a textile is soiled that has already been washed several times previously with an agent containing such oil and grease-releasing components. Preferred oil- and grease-removing components include, e.g., non-ionic cellulose ethers such as methyl cellulose and methyl hydroxypropyl cellulose with a proportion of methoxyl groups of 15 to 30 wt. % and of hydroxypropoxyl groups of 1 to 15 wt. %, in each case based on the non-ionic cellulose ether, as well as the polymers of phthalic acid and/or terephthalic acid or derivatives thereof with monomeric and/or polymeric diols known from the prior art, in particular polymers of ethylene terephthalates and/or polyethylene glycol terephthalates or anionically and/or non-ionically modified derivatives thereof. Such polymers are commercially available, e.g., under the trade name Texcare®.

Co-polymers based on polyethylene imine, polyvinyl acetate and polyethylene glycol can also be used as anti-redeposition agents.

Preferably, the composition may also contain dye transfer inhibitors, preferably in amounts of 0.1 to 2 wt. %, more preferably 0.1 to 1 wt. %, in particular preferred 0.01 to 0.1 wt. %, which in a preferred embodiment of the invention are polymers of vinyl pyrrolidone, vinyl imidazole, vinyl pyridine-N-oxide or copolymers thereof.

Graying inhibitors have the function of keeping the dirt detached from the textile fiber suspended in the liquor. Water-soluble colloids of mostly organic nature are suitable for this purpose, e.g., starch, glue, gelatin, salts of ether carboxylic acids or ether sulfonic acids of starch or cellulose, or salts of acid sulfuric acid esters of cellulose or starch. Water-soluble polyamides containing acidic groups are also suitable for this purpose. Furthermore, starch derivatives other than those mentioned above can be used, e.g., aldehyde starches. Preferably, cellulose ethers, such as carboxy methyl cellulose (Na salt), methyl cellulose, hydroxyalkyl cellulose and mixed ethers, such as methyl hydroxyethyl cellulose, methyl hydroxypropyl cellulose, methyl carboxymethyl cellulose and mixtures thereof. When incorporated into compositions according to the invention graying inhibitors are included in amounts of 0.05 to 5 wt.-%, preferably 0.1 to 2 wt. %, in particular 0.05 to 0.5 wt. %, in relation to the total weight of the composition.

It is preferred that the dye transfer inhibitor is a polymer or copolymer of cyclic amines such as vinyl pyrrolidone and/or vinyl imidazole. Polymers suitable as dye transfer inhibitors include polyvinyl pyrrolidone (PVP), polyvinyl imidazole (PVI), copolymers of vinyl pyrrolidone and vinyl imidazole (PVP/PVI), polyvinyl pyridine-N-oxide, poly-N-carboxymethyl-4-vinyl pyridium chloride, polyethylene glycol-modified copolymers of vinyl pyrrolidone and vinyl imidazole, and mixtures thereof. Polyvinyl pyrrolidone (PVP), polyvinyl imidazole (PVI) or copolymers of vinyl pyrrolidone and vinyl imidazole (PVP/PVI) are particularly preferred as dye transfer inhibitors. The polyvinyl pyrrolidones (PVP) used preferably have an average molecular weight of 2500 to 400000 and are commercially available from ISP Chemicals as PVP K 15, PVP K 30, PVP K 60 or PVP K 90 or from BASF as Sokalan® HP 50 or Sokalan® HP 53. The copolymers of vinyl pyrrolidone and vinyl imidazole (PVP/PVI) used preferably have a molecular weight in the range of 5000 to 100000 g/mol. A PVP/PVI copolymer is commercially available, e.g., from BASF as Sokalan® HP 56. Another preferred dye transfer inhibitor is polyethylene glycol-modified copolymers of vinyl pyrrolidone and vinyl imidazole, which are available, e.g., from BASF as Sokalan® HP 66.

Further soil release polymers that may be used are acrylic copolymers, e.g., the copolymer of ((2-methacryloyloxy)-ethyl)-trimethyl ammonium chloride commercially available under the trade name Sokalan® SR 400.

Suitable performance polymers also comprise polyalkoxylated polyalkylene imines, such as (poly)ethoxylated polyethylene imines. The polyalkoxylated polyalkylene imines are polymers having a polyalkylene imine backbone bearing polyalkoxy groups on the N atoms. It preferably has a weight average molecular weight Mw in the range from 5000 to 60000 g/mol, in particular from 10000 to 22500 g/mol. The polyalkylene imine has primary amino functions at its terminals and preferably both secondary and tertiary amino functions in its interior; optionally, it may also have only secondary amino functions in its interior, resulting in a linear polyalkylene imine rather than a branched chain polyalkylene imine. The ratio of primary to secondary amino groups in the polyalkylene imine is preferably in the range from 1:0.5 to 1:1.5, in particular in the range from 1:0.7 to 1:1. The ratio of primary to tertiary amino groups in the polyalkylene imine is preferably in the range from 1:0.2 to 1:1, in particular in the range from 1:0.5 to 1:0.8. Preferably, the polyalkylene imine has a weight average molecular weight in the range from 500 to 50000 g/mol, in particular from 550 to 2000 g/mol. The N atoms in the polyalkylene imine are preferably separated from one another by alkylene groups having 2 to 12 C atoms, in particular 2 to 6 C atoms, wherein not all alkylene groups need to have the same number of C atoms. Ethylene groups, 1,2-propylene groups, 1,3-propylene groups, and mixtures thereof are particularly preferred. The primary amino functions in the polyalkylene imine may carry 1 or 2 polyalkoxy groups and the secondary amino functions may carry 1 polyalkoxy group, although not every amino function has to be alkoxy group-substituted. The average number of alkoxy groups per primary and secondary amino function in the polyalkoxylated polyalkylene imine is preferably 5 to 100, in particular 10 to 50. The alkoxy groups in the polyalkoxylated polyalkylene imine are preferably ethoxy, propoxy or butoxy groups or mixtures thereof. Polyethoxylated polyethylene imines are particularly preferred. The polyalkoxylated polyalkylene imines are accessible by reacting the polyalkylene imines with epoxides corresponding to the alkoxy groups. Desirably, the terminal OH function of at least some of the polyalkoxy substituents may be replaced by an alkyl ether function having 1 to 10, in particular 1 to 3, carbon atoms. Such a polyalkoxylated polyalkylene imine is available, e.g., from BASF as Sokalan® HP 20. In various embodiments, the at least one additional ingredient comprises such polyalkoxylated polyalkylene imine, in particular (poly)ethoxylated polyethylene imine.

### Further Ingredients

In addition to the components mentioned so far, the cleaning compositions according to the invention can comprise other ingredients that further improve the practical and/or aesthetic properties of the cleaning composition. These include, e.g., additives for improving the flow and drying behavior, for adjusting the viscosity, and/or for stabilization and other auxiliary and additional substances that are customary in cleaning composition, such as UV stabilizers, perfume, pearlescent agents, dyes, corrosion inhibitors, preservatives, bitterns, organic salts, disinfectants, structuring polymers, defoamers, encapsulated ingredients (e.g., encapsulated perfume), pH adjusters and skin-feel-improving or nourishing additives.

Polymeric thickening agents within the meaning of the present invention are the polycarboxylates which have a thickening action as polyelectrolytes, preferably homo- and co-polymerizates of acrylic acid, in particular acrylic acid copolymers such as acrylic acid-methacrylic acid copolymers, and the polysaccharides, in particular heteropolysaccharides, and other conventional thickening polymers.

Suitable polysaccharides or heteropolysaccharides are the polysaccharide gums, e.g., gum arabic, agar, alginates, carrageenans and their salts, guar, guar gum, tragacanth, gellan, ramsan, dextran or xanthan and their derivatives, e.g., propoxylated guar, and mixtures thereof. Other polysaccharide thickeners, such as starches or cellulose derivatives, may alternatively or preferably be used in addition to a polysaccharide gum, e.g., starches of various origins and starch derivatives, e.g., hydroxy ethyl starch, starch phosphate esters or starch acetates, or carboxy methyl cellulose or its sodium salt, methyl, ethyl, hydroxy ethyl, hydroxy propyl, hydroxy propyl methyl or hydroxy ethyl methyl cellulose or cellulose acetate.

Acrylic acid polymers suitable as polymeric thickening agents are, e.g., high-molecular-weight homopolymers of acrylic acid (INCI: carbomer) cross-linked with a polyalkenyl polyether, in particular an allyl ether of sucrose, pentaerythritol or propylene, also referred to as carboxy vinyl polymers.

However, particularly suitable polymeric thickening agents are the following acrylic acid copolymers: (i) copolymers of two or more monomers from the group of acrylic acid, methacrylic acid and their simple esters, preferably formed with C₁₋₄ alkanols (INCI: acrylates copolymer) which include, e.g., the copolymers of methacrylic acid, butyl acrylate and methyl methacrylate (CAS 25035-69-2) or butyl acrylate and methyl methacrylate (CAS 25852-37-3); (ii) cross-linked high-molecular-weight acrylic acid copolymers, which include, e.g., the copolymers of C₁₀₋₃₀ alkyl acrylates cross-linked with an allyl ether of sucrose or pentaerythritol with one or more monomers from the group of acrylic acid, methacrylic acid and their simple esters, preferably formed by C₁₋₄ alkanols, (INCI: acrylates/C10-30 alkyl acrylate cross-polymer).

The content of polymeric thickening agent is usually not more than 8 wt. %, preferably from 0.1 to 7 wt. %, particularly preferably from 0.5 to 6 wt. %, in particular from 1 to 5 wt. % and most preferably from 1.5 to 4 wt. %, e.g., from 2 to 2.5 wt. %, based on the total weight of the composition.

To stabilize the cleaning composition according to the invention, in particular at a high surfactant content, one or more dicarboxylic acids and/or their salts can be added, in particular to a composition of Na salts of adipic, succinic and glutaric acid, e.g., as is available under the trade name Sokalan® DSC. The use here is advantageously in amounts of 0.1 to 8 wt. %, preferably 0.5 to 7 wt. %, in particular 1.3 to 6 wt. % and particularly preferably 2 to 4 wt. %, based on the total weight of the composition.

However, if the use thereof can be dispensed with, the agent according to the invention is preferably free of dicarboxylic acids (dicarboxylic acid salts).

In some embodiments, the cleaning compositions according to the invention comprise at least one chelating agent. Suitable chelating agents may include, but are not limited to copper, iron, and/or manganese chelating agents, and mixtures thereof. In some embodiments, the cleaning compositions according to the invention comprises from 0.1 to 15 wt. % or even from 3.0 to 10 wt. % chelating agent by weight of composition.

In some embodiments, the compositions according to the invention comprise at least one deposition aid. Suitable deposition aids include, but are not limited to, polyethylene glycol, polypropylene glycol, polycarboxylate, soil release polymers such as polyterephthalic acid, clays such as kaolinite, montmorillonite, attapulgite, illite, bentonite, halloysite, and mixtures thereof.

In some embodiments, the compositions according to the invention comprise one or more bleach, bleach activator, and/or bleach catalyst. In some embodiments, the compositions according to the invention comprise inorganic and/or organic bleaching compound(s). Inorganic bleaches may include, but are not limited to perhydrate salts (e.g., perborate, percarbonate, perphosphate, persulfate, and persilicate salts). In some embodiments, inorganic perhydrate salts are alkali metal salts. In some embodiments, inorganic perhydrate salts are included as the crystalline solid, without additional protection, although in some other embodiments, the salt is coated. Bleach activators are typically organic peracid precursors that enhance the bleaching action in the course of cleaning at temperatures of 60° C. and below. Bleach activators suitable for use herein include compounds which, under perhydrolysis conditions, give aliphatic peroxy carboxylic acids having preferably from about 1 to about 10 carbon atoms, in particular from about 2 to about 4 carbon atoms, and/or optionally substituted perbenzoic acid. Bleach catalysts typically include, e.g., manganese triazacyclononane and related complexes, and cobalt, copper, manganese, and iron complexes.

In some embodiments, the compositions according to the invention comprise one or more catalytic metal complex. In some embodiments, a metal-containing bleach catalyst finds use. In some embodiments, the metal bleach catalyst comprises a catalyst system comprising a transition metal cation of defined bleach catalytic activity (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylene diamine tetraacetic acid, ethylene diamine tetra-(methylene phosphonic acid) and water-soluble salts thereof are used. In some embodiments, the compositions according to the invention are catalyzed by means of a manganese compound. Such compounds and levels of use are well known in the art. In some embodiments, cobalt bleach catalysts find use in the compositions according to the invention. Various cobalt bleach catalysts are known in the art and are readily prepared by known procedures.

### Formulation

The compositions of the present invention include all solid, powdered, liquid, gel or pasty administration forms of compositions according to the invention, which may optionally also consist of a plurality of phases and can be present in compressed or uncompressed form. The agent may be present as a flowable powder, in particular having a bulk density of from 300 to 1200 g/L, in particular from 500 to 900 g/L or from 600 to 850 g/L. The solid administration forms of the compositions also include extrudates, granules, tablets or pouches. Alternatively, the composition may also be in liquid, gel or pasty form, e.g., in the form of a non-aqueous liquid washing agent or a non-aqueous paste or in the form of an aqueous liquid washing agent or a water-containing paste. The composition may also be present as a one-component system. Such compositions consist of one phase. Alternatively, an agent may also consist of a plurality of phases. Such a composition is therefore divided into a plurality of components (multi-component system).

The compositions according to the invention are typically formulated such that, during use in aqueous cleaning operations, the wash liquor will have a pH of from 3.0 to 11, as measured in 1 wt. % aqueous solution at 20° C. Liquid product formulations are typically formulated to have a pH from 5.0 to 9.0, more preferably from 7.5 to 9. Granular laundry products are typically formulated to have a pH from 8.0 to 11.0. Techniques for controlling pH at recommended usage levels include the use of buffers, alkalis, acids, etc., and are well known to those skilled in the art.

Suitable high pH cleaning compositions typically have a pH of from 9.0 to 11.0, or even a pH of from 9.5 to 10.5, as measured in 1 wt. % aqueous solution at 20° C. Such cleaning compositions typically comprise a sufficient amount of a pH modifier, such as sodium hydroxide, monoethanol amine, or hydrochloric acid, to provide such cleaning composition with a pH of from 9.0 to 11.0. Such compositions typically comprise at least one base-stable enzyme. In some embodiments, the compositions are liquids, while in other embodiments, they are solids.

In one embodiment, the cleaning compositions according to the invention include those having a pH of from 7.4 to 11.5, or pH 7.4 to 11.0, or pH 7.5 to 11.5, or pH 7.5 to 11.0, or pH 7.5 to 10.5, or pH 7.5 to 10.0, or pH 7.5 to 9.5, or pH 7.5 to 9.0, or pH 7.5 to 8.5, or pH 7.5 to 8.0, or pH 7.6 to 11.5, or pH 7.6 to 11.0, or pH 7.6 to 10.5, or pH 8.7 to 10.0, or pH 8.0 to 11.5, or pH 8.0 to 11.0, or pH 8.0 to 10.5, or pH 8.0 to 10.0; as measured in 1 wt. % aqueous solution at 20° C. In various other embodiments, in particular if the composition is a fabric conditioning composition, the pH may be lower and may be in the range of 5.0 to 7.5, as measured in 1 wt. % aqueous solution at 20° C.

Concentrations of detergent compositions in typical wash solutions throughout the world vary from less than about 800 ppm of detergent composition (“low detergent concentration geographies”), e.g., about 667 ppm in Japan, to between about 800 ppm to about 2000 ppm (“medium detergent concentration geographies”), e.g., about 975 ppm in U.S. and about 1500 ppm in Brazil, to greater than about 2000 ppm (“high detergent concentration geographies”), e.g., about 4500 ppm to about 5000 ppm in Europe and about 6000 ppm in high suds phosphate builder geographies.

In some embodiments, the cleaning compositions according to the invention may be utilized at a temperature of from 10 to 60° C., or 20 to 60° C., or 30 to 60° C., 40 to 60° C., from about 40 to 55° C., or all ranges within 10 to 60° C. In some embodiments, the detergent compositions according to the invention are used in “cold water washing” at temperatures of from 10 to 40° C., or from 20 to 30° C., from 15 to 25° C., from 15 to 35° C., or all ranges within 10 to 40° C.

As a further example, different geographies typically have different water hardness. Water hardness is usually described in terms of the grains per gallon mixed Ca²⁺/Mg²⁺. Hardness is a measure of the amount of calcium (Ca²⁺) and magnesium (Mg²⁺) in the water. Most water in the United States is hard, but the degree of hardness varies. Moderately hard (60 to 120 ppm) to hard (121 to 181 ppm) water has 60 to 181 ppm (parts per million converted to grains per U.S. gallon is ppm #divided by 17.1 equals grains per gallon) of hardness minerals.

TABLE I
Water Hardness Levels

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

European water hardness is typically greater than about 10.5 (e.g., 10.5 to 20.0) grains per gallon mixed Ca²⁺/Mg²⁺ (e.g., about 15 grains per gallon mixed Ca²⁺/Mg²⁺). North American water hardness is typically greater than Japanese water hardness, but less than European water hardness. For example, North American water hardness can be between about 3 to about 10 grains, about 3 to about 8 grains or about 6 grains. Japanese water hardness is typically lower than North American water hardness, usually less than about 4, e.g., about 3 grains per gallon mixed Ca²⁺/Mg²⁺.

### Methods and Uses

Another aspect of the present invention is a method for the cleaning or conditioning of textiles or fabrics, which is characterized in that in at least one method step, a composition according to the invention is used, in particular contacted with the fabric or textile. The fabrics/textiles preferably are selected from the group consisting of: a textile or fabric comprising or consisting of polyester, and wherein the polyester is preferably selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof, more preferably selected from the group consisting of polyethylene terephthalate (PET), polytrimethylene terephthalate (PTT), polybutylene terephthalate (PBT), and combinations thereof.

In various embodiments, the method described above is characterized in that the composition according to the invention is used at a temperature of from 0 to 100° C., preferably 0 to 80° C., more preferably 30 to 60° C., even more preferably 20 to 40° C. and most preferably at 20° C., 30° C. or 40° C.

These include both manual and mechanical methods, with mechanical methods being preferred. Methods for cleaning and/or conditioning of textiles are generally characterized by the fact that, in a plurality of method steps, various (cleaning) active substances are applied to the material to be cleaned/conditioned and washed off after the exposure time, or in that the material to be cleaned/conditioned is otherwise treated with a washing/conditioning agent or a solution or dilution of this agent. All conceivable washing or cleaning or conditioning methods can be enhanced in at least one of the method steps by the use of a composition according to the invention, and therefore represent embodiments of the present invention.

All aspects, objects and embodiments described for the compositions according to the invention are also applicable to this subject matter of the invention relating to methods of use thereof. Therefore, reference is expressly made at this point to the disclosure at the appropriate point with the note that this disclosure also applies to the above-described methods according to the invention.

Since enzymes naturally already have catalytic activity and also exhibit this in media which otherwise have no cleaning power, e.g., in a simple buffer, a single and/or the sole step of such a method can consist in at least one lipolytic enzyme having polyesterase activity, which is the only cleaning/conditioning active component, being brought into contact with the textile or fabric, preferably in a buffer solution or in water. This constitutes a further embodiment of this subject matter of the invention.

Alternative embodiments of this subject matter of the invention are also represented by methods for treating textile raw materials or for textile care, in which a composition according to the invention becomes active in at least one method step. Among these, methods for textile raw materials, fibers or textiles with synthetic constituents are preferred, and very particularly for those with polyester or blends thereof, e.g., polyester-cotton blends or polyamide-cotton blends.

Further, the invention also relates to the use of at least one variant lipolytic enzyme having polyesterase activity described herein or compositions as described herein for adjusting, in particular improving, the thermophysiological properties of the fabric or textile thus treated. The properties comprise heat and moisture management, in particular moisture management, and relate to the absorbency of water of the fibers thus treated. Another related use is increasing the hydrophilicity of the fibers thus treated. Without wishing to be bound to any particular theory, the inventors believe that treating the textile/fabric surface with the polyesterase leads to changes of the surface that increase hydrophilicity, which in turn results in improved water absorbency. In various embodiments of these uses, the at least one variant lipolytic enzyme having polyesterase activity is comprised in the composition in an amount of from 0.00001 to 1 wt. %, preferably in an amount of from 0.0001 to 0.5 wt. %, particularly preferably in an amount of from 0.001 to 0.1 wt. % In further various embodiments, the at least one variant lipolytic enzyme having polyesterase activity, are applied to textiles, wherein the textiles are selected from the group consisting of:
a textile or fabric comprising or consisting of polyester, and wherein the polyester is preferably selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof, more preferably selected from the group consisting of polyethylene terephthalate (PET), polytrimethylene terephthalate (PTT), polybutylene terephthalate (PBT), and combinations thereof, or

All aspects, objects and embodiments described for the cleaning compositions according to the invention are also applicable to this subject matter of the invention. Therefore, reference is expressly made at this point to the disclosure at the appropriate point with the note that this disclosure also applies to the above-described uses according to the invention.

In some embodiments, a polyester (e.g., PET)-containing textile or fabric may have a hydrolysable polymer end or a loop on their surface. The lipolytic enzymes having polyesterase activity described herein find use in surface modification of polyester (e.g., PET) fibers, which may improve factors such as finishing fastness, dyeability, wettability, anti-graying and de-pilling. In some embodiments, polymer chains that protrude or form a loop on the surface of a polyester (e.g., PET)-containing textile, fiber or film may be hydrolyzed by the lipolytic enzymes having polyesterase activity described herein to carboxylic acid and hydroxyl residues, thus increasing surface hydrophilicity. Pilling is the formation of small, fuzzy balls on the surface of polyester (e.g., PET) fabrics resulting in an unsightly worn appearance of the textile. Generally, these nodules are produced by loose fibers in the fabric or those which have been released from the tissue.

The textile or fabric can be contacted with at least one variant lipolytic enzyme having polyesterase activity described herein or a composition comprising at least one variant lipolytic enzyme having polyesterase activity described herein in a washing machine or in a manual wash tub (e.g., for handwashing). In one embodiment, the textile or fabric is contacted with at least one variant lipolytic enzyme having polyesterase activity described herein or a composition comprising at least one variant lipolytic enzyme having polyesterase activity described herein in a wash liquor. In another embodiment, a solution containing at least one variant lipolytic enzyme having polyesterase activity described herein is incubated with or flowed over the polyester- or polyester-cotton-blend-containing material, such as by pumping the solution through tubing or pipes or by filling a reservoir with the solution.

In some embodiments, the textiles or articles are contacted with at least one variant lipolytic enzyme having polyesterase activity described herein or a composition comprising at least one variant lipolytic enzyme having polyesterase activity described herein under conditions having a temperature that allows for activity of the variant lipolytic enzyme. In some embodiments, the temperature in the methods disclosed herein include those between 10 to 60° C., 10 to 45° C., 15 to 55° C., 15 to 50° C., 15 to 45° C., 20 to 60° C., 20 to 50° C. and 20 to 45° C.

Other aspects and embodiments of the present compositions and methods will be apparent from the foregoing description and following examples. Various alternative embodiments beyond those described herein can be employed in practicing the invention without departing from the spirit and scope of the invention. Accordingly, the claims, and not the specific embodiments described herein, define the scope of the invention and as such methods and structures within the scope of the claims and their equivalents are covered thereby.

### Embodiments

1. A cleaning or fabric conditioning composition, comprising
(a) at least one variant lipolytic enzyme, wherein said variant lipolytic enzyme comprises an amino acid sequence having at least 70% identity to the full length amino acid sequence of SEQ ID NO:2, comprising the substitutions T064V-T117L-T177N/R-I178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161H, G175A/E, F207L/T, V210I, Q227H, A236P, S244E, E254Q, and R256K, wherein the positions are numbered by reference to the amino acid sequence of SEQ ID NO:2, and wherein the variant has esterase activity; and
(b) at least one additional ingredient selected from the group consisting of complexing agents, surfactants, performance polymers and combinations thereof.

2. The cleaning or fabric conditioning composition of embodiment 1, wherein the variant lipolytic enzyme comprises an amino acid sequence having at least 75%, 80%, 85%, 90%, 91%, 92%, 93%, 94%, 95%, 96%, 97%, 98%, or 99% identity to the full-length amino acid sequence of SEQ ID NO:2.

3. The cleaning or fabric conditioning composition of embodiment 1 or 2, wherein the variant lipolytic enzyme is derived from a parent enzyme comprising an amino acid sequence having at least 75%, 80%, 85%, 90%, 91%, 92%, 93%, 94%, 95%, 96%, 97%, 98%, or 99% identity to the full-length amino acid sequence of SEQ ID NO:2.

4. The cleaning or fabric conditioning composition of any preceding embodiment, wherein the variant lipolytic enzyme comprises a combination of substitutions selected from the group consisting of R40T-T64V-T117L-G175E-T177N-F180P-Y182A-R190L-S205G-F207L-S212D-F226L-Y239I-L249P-S252I-L258F, R40T-G61D-T64V-S70E-T117L-T177N-I178L-F180P-Y182A-R190L-S205G-F207T-S212D-F226L-Q227H-A236P-Y239I-L249P-S252I-E254Q-L258F, R40T-T64V-S70E-T117L-T177N-I178L-F180P-Y182A-R190L-S205G-F207T-S212D-F226L-A236P-Y239I-L249P-S252I-E254Q-L258F, R40A-T64V-S70E-T117L-T177N-I178L-F180P-Y182A-R190L-S205G-F207T-S212D-F226L-A236P-Y239I-L249P-S252I-E254Q-L258F, R40T-T64V-S70E-T117L-Q161H-T177N-I178L-F180P-Y182A-R190L-S205G-F207T-S212D-F226L-A236P-Y239I-L249P-S252I-E254Q-L258F, R40T-T64V-S70E-T117L-G175A-T177N-I178L-F180P-Y182A-R190L-S205G-F207T-S212D-F226L-A236P-Y239I-L249P-S252I-E254Q-L258F, R40T-T64V-S70E-T117L-T177N-I178L-F180P-Y182A-R190L-S205G-F207T-V210I-S212D-F226L-A236P-Y239I-L249P-S252I-E254Q-L258F, R40T-T64V-S70E-T117L-T177N-I178L-F180P-Y182A-R190L-S205G-F207T-S212D-F226L-A236P-Y239I-S244E-L249P-S252I-E254Q-L258F, R40T-T64V-S70E-T117L-T177N-I178L-F180P-Y182A-R190L-S205G-F207T-S212D-F226L-A236P-Y239I-L249P-S252I-E254Q-R256K-L258F, V14S-R40A-G59Y-G61D-T64V-A66D-S70E-T117L-Q161H-T177R-I178L-F180P-Y182A-R190L-S205G-F207T-V210I-S212D-F226L-A236P-Y239I-L249P-S252I-E254Q-R256K-L258F, V14S-R40A-G59Y-G61D-T64V-S70E-T117L-Q161H-T177R-I178L-F180P-Y182A-R190L-S205G-F207T-V210I-S212D-F226L-A236P-Y239I-L249P-S252I-E254Q-R256K-L258F, R40T-G61D-T64V-S70E-T117L-Q161H-T177R-I178L-F180P-Y182A-R190L-S205G-F207T-V210I-S212D-F226L-A236P-Y239I-L249P-S252T-E254Q-R256K-L258F, and V14S-R40A-G59Y-G61D-T64V-A66D-S70E-T117L-Q161H-G175A-T177R-I178L-F180P-Y182A-R190L-S205G-F207T-V210I-S212D-F226L-A236P-Y239I-L249P-S252I-E254Q-R256K-L258F, wherein the positions are numbered by reference to the amino acid sequence of SEQ ID NO:2.

5. The cleaning or fabric conditioning composition of any preceding embodiment, wherein the variant lipolytic enzyme has lipolytic activity on a polyester selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof.

6. The cleaning or fabric conditioning composition of any preceding embodiment, wherein the variant lipolytic enzyme is contained in the composition in an amount of from 0.00001 to 1 wt. %, preferably in an amount of from 0.0001 to 0.5 wt. %, particularly preferably in an amount of from 0.001 to 0.1 wt. %.

7. The cleaning or fabric conditioning composition of any preceding embodiment, wherein the at least one additional ingredient comprises a performance polymer.

8. The cleaning or fabric conditioning composition of embodiment 7, wherein the performance polymer is an alkoxylated polyethylene imine.

9. The cleaning or fabric conditioning composition of any preceding embodiment, wherein the at least one additional ingredient comprises a complexing agent.

10. The cleaning or fabric conditioning composition of any preceding embodiment, wherein the cleaning or fabric conditioning composition comprises at least one further ingredient selected from the group consisting of builders, bleaching agents, bleach activators, water-miscible organic solvents, sequestering agents, electrolytes, pH regulators, optical brighteners, graying inhibitors, foam regulators, dyes and fragrances and combinations thereof.

11. The cleaning or fabric conditioning composition of any preceding embodiment, wherein the cleaning or fabric conditioning composition has a pH of 7.0 to 11.0, as measured in 1 wt. % aqueous solution at 20° C.

12. The cleaning or fabric conditioning composition of any preceding embodiment, wherein the cleaning or fabric conditioning composition is present in liquid form.

13. The cleaning or fabric conditioning composition of any preceding embodiment, wherein the cleaning or fabric conditioning composition is in unit dose form.

14. A method of cleaning or conditioning a textile or fabric, comprising:
a) providing a composition comprising at least one variant lipolytic enzyme, wherein said variant lipolytic enzyme comprises an amino acid sequence having at least 70% identity to the full length amino acid sequence of SEQ ID NO:2, comprising the substitutions T064V-T117L-T177N/R-I178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161H, G175A/E, F207L/T, V210I, Q227H, A236P, S244E, E254Q, and R256K, wherein the positions are numbered by reference to the amino acid sequence of SEQ ID NO:2, and wherein the variant has esterase activity or a composition according to any one of embodiments 1-13; and
b) contacting the textile or fabric with the composition, wherein the textile or fabric comprises or consists of polyester, and wherein the polyester is preferably selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof, more preferably selected from the group consisting of polyethylene terephthalate (PET), polytrimethylene terephthalate (PTT), polybutylene terephthalate (PBT), and combinations thereof.

15. Method for improving the thermophysiological properties of a textile or fabric comprising or consisting of polyester, the method comprising
a) providing a composition comprising at least one variant lipolytic enzyme, wherein said variant lipolytic enzyme comprises an amino acid sequence having at least 70% identity to the full length amino acid sequence of SEQ ID NO:2, comprising the substitutions T064V-T117L-T177N/R-I178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161H, G175A/E, F207L/T, V210I, Q227H, A236P, S244E, E254Q, and R256K, wherein the positions are numbered by reference to the amino acid sequence of SEQ ID NO:2, and wherein the variant has esterase activity or a composition according to any one of embodiments 1-13; and
b) contacting the fabric or textile with the composition.

16. Method according to embodiment 15, wherein the thermophysiological properties comprise heat and moisture management.

17. Method according to embodiment 15, wherein the thermophysiological properties comprise wear comfort.

18. Method for increasing the hydrophilicity of a textile or fabric comprising or consisting of polyester, the method comprising
a) providing a composition comprising at least one variant lipolytic enzyme, wherein said variant lipolytic enzyme comprises an amino acid sequence having at least 70% identity to the full length amino acid sequence of SEQ ID NO:2, comprising the substitutions T064V-T117L-T177N/R-I178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161H, G175A/E, F207L/T, V210I, Q227H, A236P, S244E, E254Q, and R256K, wherein the positions are numbered by reference to the amino acid sequence of SEQ ID NO:2, and wherein the variant has esterase activity or a composition according to any one of embodiments 1-13; and
b) contacting the fabric or textile with the composition.

19. The method according to any one of embodiments 14 to 18, wherein the polyester is preferably selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof, more preferably selected from the group consisting of polyethylene terephthalate (PET), polytrimethylene terephthalate (PTT), polybutylene terephthalate (PBT), and combinations thereof.

20. The method according to any one of embodiments 14-19, further comprising a rinsing step.

### EXAMPLES

### Example 1: Recombinant Expression and Generation of P. mendocina Lipase Variants

A synthetic, codon-optimized gene (SEQ ID NO:1) encoding the wild-type Pseudomonas mendocina lipase (SEQ ID NO:2) was made and served as template for the construction of plasmids expressing variant polypeptides thereof. Lipase genes were produced by either GeneArt AG (Regensburg, Germany) or Twist Bioscience (San Francisco, USA) and cloned into the pSB expression vector (Babé, L. M., et al., Biotechnol Appl Biochem. 27: 117-124, 1998) using standard molecular biology techniques resulting in expression plasmids suitable for expression in Bacillus subtilis. Elements of the constructs included: DNA fragments comprising the aprE promoter sequence (SEQ ID NO:3), the nucleotide sequence encoding either the aprE signal peptide sequence (SEQ ID NO:4) or a hybrid aprE-P. mendocina lipase signal peptide sequence (SEQ ID NO:5), the sequence corresponding to the gene encoding a mature lipase, the BPN′ terminator (SEQ ID NO:6), and additional elements from pUB110 (McKenzie et al., Plasmid 15: 93-103, 1986) including a replicase gene (reppUB), a neomycin/kanamycin resistance gene (neo), and a bleomycin resistance marker (bleo).

A suitable B. subtilis host strain was transformed with the pSB expression plasmid using a method known in the art (WO 2002/014490). The transformation mixtures were plated onto LA plates containing 10 ppm neomycin sulfate and incubated overnight at 37° C. Single colonies were picked and grown in Luria broth at 37° C. under antibiotic selection.

To generate the enzyme samples for screening, the transformed B. subtilis cells were grown in 96 well microtiter plates (MTPs) at 37° C. for 68 hours in cultivation medium (enriched semi-defined media based on MOPS buffer, with urea as the major nitrogen source, glucose as the main carbon source, and supplemented with 1% soy tone for robust cell growth) in each well. Cultures were harvested by centrifugation at 3600 rpm for 15 min and filtered through Multiscreen® filter plates (EMD Millipore, Billerica, MA, USA) using a Millipore vacuum system. The filtered culture supernatants were used for the assays described below. Typically, the culture broth was diluted in 100 mM Tris pH 8 in 96 well plate (Nunc, 267245). Enzyme concentration was determined by separation of protein components using a Zorbax 300 SB-C3 column (Agilent) and running a linear gradient of 0.1% Trifluoroacetic acid in water (Buffer A) and 0.1% Trifluoroacetic acid in Acetonitrile (Buffer B) with detection at 220 nm column on UHPLC. The enzyme concentration of the samples was calculated using a standard curve of the purified reference enzyme PEV 132.

### Example 2: Enzyme Activity of P. Mendocina Lipase Variants

The enzymatic activity of P. mendocina lipase variants (listed in Table 1) was tested on PET (polyethylene terephthalate) substrate by measuring the hydrolysis of PET pellet substrate in solution. PET pellets were purchased from Scientific Polymer Products (Cat #138). One PET pellet (20-30 mg) was added to each well of a microtiter plate (Nunc, 267245) and detergent solutions were added. Specifically, the detergent solution consists of two hundred microliters of Formula A (3.0 g/L) (composition in Table 2) prepared in 10 mM Tris-HCl buffer with 6 gpg water hardness Ca:Mg=3:1, pH 8.

TABLE 1
List of variants constructed with sample ID and amino acid substitutions
with respect to P. mendocina wild type enzyme

| sample ID | Amino acid substitution with respect to P. mendocina wild-type lipase |
|---|---|
| PEV132 | R40T-T64V-T117L-T177N-I178L-F180P-Y182A-R190L-S205G- |
| | F207T-S212D-F226L-Y239I-L249P-S252I-L258F |
| PEV132v1 | R040A-T064V-T117L-T177N-I178L-F180P-Y182A-R190L-S205G- |
| | F207T-S212D-F226L-Y239I-L249P-S252I-L258F |
| PEV132v2 | R040T-T064V-T117L-T177R-I178L-F180P-Y182A-R190L-S205G- |
| | F207T-S212D-F226L-Y239I-L249P-S252I-L258F |
| PEV176 | R40T-T64V-T117L-G175E-T177N-F180P-Y182A-R190L-S205G- |
| | F207L-S212D-F226L-Y239I-L249P-S252I-L258F |
| PEV192 | R40T-G61D-T64V-S70E-T117L-T177N-I178L-F180P-Y182A-R190L- |
| | S205G-F207T-S212D-F226L-Q227H-A236P-Y239I-L249P-S252I- |
| | E254Q-L258F |
| PEV194 | R40T-T64V-S70E-T117L-T177N-I178L-F180P-Y182A-R190L- |
| | S205G-F207T-S212D-F226L-A236P-Y239I-L249P-S252I-E254Q- |
| | L258F |
| PEV304 | R40A-T64V-S70E-T117L-T177N-I178L-F180P-Y182A-R190L- |
| | S205G-F207T-S212D-F226L-A236P-Y239I-L249P-S252I-E254Q- |
| | L258F |
| PEV314 | R40T-T64V-S70E-T117L-Q161H-T177N-I178L-F180P-Y182A- |
| | R190L-S205G-F207T-S212D-F226L-A236P-Y239I-L249P-S252I- |
| | E254Q-L258F |
| PEV315 | R40T-T64V-S70E-T117L-G175A-T177N-I178L-F180P-Y182A- |
| | R190L-S205G-F207T-S212D-F226L-A236P-Y239I-L249P-S252I- |
| | E254Q-L258F |
| PEV318 | R40T-T64V-S70E-T117L-T177N-I178L-F180P-Y182A-R190L- |
| | S205G-F207T-V210I-S212D-F226L-A236P-Y239I-L249P-S252I- |
| | E254Q-L258F |
| PEV319 | R40T-T64V-S70E-T117L-T177N-I178L-F180P-Y182A-R190L- |
| | S205G-F207T-S212D-F226L-A236P-Y239I-S244E-L249P-S252I- |
| | E254Q-L258F |
| PEV320 | R40T-T64V-S70E-T117L-T177N-I178L-F180P-Y182A-R190L- |
| | S205G-F207T-S212D-F226L-A236P-Y239I-L249P-S252I-E254Q- |
| | R256K-L258F |
| PEV325 | V14S-R40A-G59Y-G61D-T64V-A66D-S70E-T117L-Q161H-T177R- |
| | I178L-F180P-Y182A-R190L-S205G-F207T-V210I-S212D-F226L- |
| | A236P-Y239I-L249P-S252I-E254Q-R256K-L258F |
| PEV328 | V14S-R40A-G59Y-G61D-T64V-S70E-T117L-Q161H-T177R-I178L- |
| | F180P-Y182A-R190L-S205G-F207T-V210I-S212D-F226L-A236P- |
| | Y239I-L249P-S252I-E254Q-R256K-L258F |
| PEV329 | R40T-G61D-T64V-S70E-T117L-Q161H-T177R-I178L-F180P-Y182A- |
| | R190L-S205G-F207T-V210I-S212D-F226L-A236P-Y239I-L249P- |
| | S252I-E254Q-R256K-L258F |
| PEV334 | V14S-R40A-G59Y-G61D-T64V-A66D-S70E-T117L-Q161H-G175A- |
| | T177R-I178L-F180P-Y182A-R190L-S205G-F207T-V210I-S212D- |
| | F226L-A236P-Y239I-L249P-S252I-E254Q-R256K-L258F |

TABLE 2
Composition of custom-made Detergent Formula
A (HDL being prepared without polyesterase)

| | | Formula A |
|---|---|---|
| | wt. % of active | wt. % of active |
| | substance in the | substance in the |
| Chemical name | raw material | formulation |
| Water demineralized | 100 | Ad 100 |
| Alkyl benzene sulfonic | 96 | 3-7 |
| acid (LAS) | | |
| Anionic surfactant | 70 | 2-6 |
| (FAEOS) | | |
| Coconut fatty acid | 30 | 0.3-1 |
| Non-ionic surfactant | 100 | 3-7 |
| (FAEO) | | |
| Citric acid | 100 | 0.1-2 |
| NaOH | 50 | 0.3-1 |
| Glycerol | 99.5 | 0.3-1 |
| Preserving agent | 100 | 0.05-0.1 |
| Stabilizer | 100 | 0.3-1 |
| Optical Brightener | 90 | 0.01-0.08 |
| Thickener | 25 | 1-3 |
| Soil release polymer | 30 | 0 |
| Dye transfer inhibitor | 30 | 0 |
| DTPMP | 40 | 0.05-0.2 |
| Dye, Perfume, Antifoam, | t.q. | minors |
| Enzymes (if any) | | |
| | | pH = 8.2 to 8.6 |
| | | Dosage = 50 ml |

A set of plates without PET in the well were also set up to serve as controls for enzyme background. Twenty microliters of each enzyme sample were added per well of the assay plate to initiate the reaction. The reaction was carried out at 40° C. for 24 hours with shaking (180 rpm) in incubation shaker (Infors HT, Multitron). After incubation, 100 μl of the reaction supernatant was transferred into a new UV-transparent plate (Corning 3635) and measured at 240 nm on Microplate reader (Molecular devices, SpectraMax plus 384). The resulting absorbance after subtracting absorbance from enzyme background plate was taken as a measure of PET hydrolysis activity. The absorbance values were plotted against enzyme concentration. Each variant was assayed in triplicates. PET activity is reported as Performance Index (PI) values, which were calculated by dividing the PET activity of each variant by that of the parent, tested at the same protein concentration. Table 3 shows the polyesterase activity on PET substrate (performance index) of variants from Table 1. Theoretical values for the PET activity of the parent enzyme at the relevant protein concentrations were calculated using the parameters extracted from a Langmuir fit of measured values from a standard curve of the parent enzyme activity.

TABLE 3
PET Activity of P. mendocina lipase variants in Formula A. Relative
activity on PET substrate is reported as Performance Index values (PI)
calculated relative to P. mendocina lipase PEV132

| sample ID | Polyesterase Activity (PI) |
|---|---|
| PEV132 | 1.0 |
| PEV132v1 | 1.1 |
| PEV132v2 | 1.0 |
| PEV318 | 1.4 |
| PEV319 | 1.5 |
| PEV314 | 1.7 |
| PEV194 | 1.8 |
| PEV329 | 1.8 |
| PEV192 | 1.9 |
| PEV328 | 1.9 |
| PEV315 | 1.9 |
| PEV334 | 1.9 |
| PEV325 | 2.1 |
| PEV320 | 2.2 |
| PEV304 | 2.2 |

### Example 3: Thermal Stability of P. mendocina Variants

The stability of P. mendocina lipase variants (shown in Table 1) was tested under stress condition in a 50% (v/v) aqueous solution of Formula A detergent by measuring the residual activity of samples after incubation at 56° C. for 16 hours. A 67% (v/v) aqueous solution of the detergent was prepared and enzyme samples from filtered culture supernatants were mixed with the appropriate volume of this detergent solution to achieve 50% (v/v) final detergent concentration. To measure the initial (unstressed) activity, aliquots of this mixture were immediately diluted in 100 mM Tris-HCl, 0.1% Triton X-100, pH 8 and assayed for activity on pNB substrate. pNB substrate (4-nitropheyl butyrate, Sigma) solution (1 mM) was prepared by adding 0.2 ml of pNB stock solution (100 mM in DMSO) to 20 ml of buffer (100 mM Tris-HCl, 0.1% Triton X-100, pH 8). Ten microliters of diluted enzyme solution were mixed into 190 μl of 1 mM pNB in assay buffer in 96 well plate (Costar, #9017, ThermoFisher) to start the reaction. The plate was mixed thoroughly, and absorbance was monitored at OD 405 nm every 12 seconds for 3 minutes in a Microplate reader (Molecular Devices, SpectraMax plus 384). The Vmax in mOD/min value of a sample not containing enzyme (blank) was subtracted from Vmax values of the enzyme-containing samples. The resulting Vmax in mOD/min was recorded as enzyme activity on pNB substrate. Once stressed and unstressed activity values were measured by hydrolysis of pNB substrate as described above, the percent (%) residual activities were calculated by taking a ratio of the stressed to unstressed activity and multiplying by 100. Table 4 shows the % residual activity of the P. mendocina lipase variants tested.

TABLE 4
Stability of P. mendocina lipase variants in Formula
A. Stability is reported as % residual activity remaining
after incubation at 56° C. for 16 hours.

| sample ID | % Residual activity |
|---|---|
| PEV132 | 20 |
| PEV176 | 90 |
| PEV192 | 20 |
| PEV194 | 40 |
| PEV304 | 40 |
| PEV314 | 50 |
| PEV315 | 50 |
| PEV318 | 60 |
| PEV319 | 80 |
| PEV320 | 100 |
| PEV325 | 90 |
| PEV328 | 100 |
| PEV329 | 80 |
| PEV334 | 90 |

### Example 4: Absorbency Test

A liquid commercial laundry detergent matrix was used for a washing test:

TABLE 5
Composition of a commercial heavy duty liquid laundry detergent

| | Wt. % of active substance |
|---|---|
| Chemical name | in the formulation |
| Water demin. | Ad 100 |
| Anionic surfactant (LAS, SLES, FS) | 5-30 |
| amphoteric surfactants | 0-5 |
| Non-ionic surfactants | 1-15 |
| Citric acid | 0-4 |
| Performance polymers | 0-5 |
| Solvent (Propandiol/EtOH) | 1-20 |
| Stabilizer | 0-1.5 |
| Optical Brightener | 0-0.2 |
| Antifoam | 0-0.2 |
| Complex builder (EDTA) | 0.2-2 |
| Dye, Perfume, Enzymes (except | minors |
| Polyesterase) | |
| pH = 8.2 to 8.6 | |
| Dosage = 46-59 mL | |

The wicking test according to DIN 53924 for determining the absorbency of the textiles was conducted as follows:

In a typical top loader automatic washing machine (Kenmore 80 series) textiles made of a blend of polyester and cotton, (1) Knitwear T-shirt, single Jersey, 50/50 (2) plain weave, 50/50, were washed with the “normal” washing program at 28° C. 10-times. Water hardness was 6,72° dH.

TABLE 6
Determining the water absorbency of textiles (capillary height
method) after washing for 10 times. All values are height
of the capillary water column in mm after 10 minutes:

| | Knitwear T-shirt | plain weave, | |
|---|---|---|---|
| Composition | 50% CO/50% PES | 50% CO/50% PES | |
| without polyesterase | 119.5 mm | 82 | mm |
| with 10 mg polyesterase | 122.5 mm | 82.5 | mm |
| with 7.5 mg polyesterase | 125.0 mm | 84.5 | mm |

The polyesterase according to the invention was shown to improve water absorbency of the textiles.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

1. A cleaning or fabric conditioning composition, comprising
(a) at least one variant lipolytic enzyme, wherein said variant lipolytic enzyme comprises an amino acid sequence having at least 70% identity to the full length amino acid sequence of SEQ ID NO:2, comprising the substitutions T064V-T117L-T177N/R-I178L-F180P-Y182A-R190L-S205G-S212D-F226L-Y239I-L249P-S252I-L258F, and further comprising at least one additional substitution selected from the group consisting of V014S, R040A/T, G059Y, G061 D, A066D, S070E, Q161 H, G175A/E, F207L/T, V210I, Q227H, A236P, S244E, E254Q, and R256K, wherein the positions are numbered by reference to the amino acid sequence of SEQ ID NO:2, and wherein the variant has esterase activity; and
(b) at least one additional ingredient selected from the group consisting of performance polymers, complexing agents, surfactants, and combinations thereof.

2. The cleaning or fabric conditioning composition of claim 1, wherein the variant lipolytic enzyme comprises an amino acid sequence having at least 75%, 80%, 85%, 90%, 91%, 92%, 93%, 94%, 95%, 96%, 97%, 98%, or 99% identity to the full-length amino acid sequence of SEQ ID NO:2.

3. The cleaning or fabric conditioning composition of claim 1, wherein the variant lipolytic enzyme is derived from a parent enzyme comprising an amino acid sequence having at least 75%, 80%, 85%, 90%, 91%, 92%, 93%, 94%, 95%, 96%, 97%, 98%, or 99% identity to the full-length amino acid sequence of SEQ ID NO:2.

4. The cleaning or fabric conditioning composition of claim 1, wherein the variant lipolytic enzyme comprises a combination of substitutions selected from the group consisting of R40T-T64V-T117L-G175E-T177N-F180P-Y182A-R190L-S205G-F207L-S212D-F226L-Y239I-L249P-S252I-L258F, R40T-G61 D-T64V-S70E-T117L-T177N-I178L-F180P-Y182A-R190L-S205G-F207T-S212D-F226L-Q227H-A236P-Y239I-L249P-S252I-E254Q-L258F, R40T-T64V-S70E-T117L-T177N-I178L-F180P-Y182A-R190L-S205G-F207T-S212D-F226L-A236P-Y239I-L249P-S252I-E254Q-L258F, R40A-T64V-S70E-T117L-T177N-I178L-F180P-Y182A-R190L-S205G-F207T-S212D-F226L-A236P-Y239I-L249P-S252I-E254Q-L258F, R40T-T64V-S70E-T117L-Q161 H-T177N-I178L-F180P-Y182A-R190L-S205G-F207T-S212D-F226L-A236P-Y239I-L249P-S252I-E254Q-L258F, R40T-T64V-S70E-T117L-G175A-T177N-I178L-F180P-Y182A-R190L-S205G-F207T-S212D-F226L-A236P-Y239I-L249P-S252I-E254Q-L258F, R40T-T64V-S70E-T117L-T177N-I178L-F180P-Y182A-R190L-S205G-F207T-V210I-S212D-F226L-A236P-Y239I-L249P-S252I-E254Q-L258F, R40T-T64V-S70E-T117L-T177N-I178L-F180P-Y182A-R190L-S205G-F207T-S212D-F226L-A236P-Y239I-S244E-L249P-S252I-E254Q-L258F, R40T-T64V-S70E-T117L-T177N-I178L-F180P-Y182A-R190L-S205G-F207T-S212D-F226L-A236P-Y239I-L249P-S252I-E254Q-R256K-L258F, V14S-R40A-G59Y-G61 D-T64V-A66D-S70E-T117L-Q161 H-T177R-I178L-F180P-Y182A-R190L-S205G-F207T-V210I-S212D-F226L-A236P-Y239I-L249P-S252I-E254Q-R256K-L258F, V14S-R40A-G59Y-G61 D-T64V-S70E-T117L-Q161 H-T177R-I178L-F180P-Y182A-R190L-S205G-F207T-V210I-S212D-F226L-A236P-Y239I-L249P-S252I-E254Q-R256K-L258F, R40T-G61 D-T64V-S70E-T117L-Q161 H-T177R-I178L-F180P-Y182A-R190L-S205G-F207T-V210I-S212D-F226L-A236P-Y239I-L249P-S252I-E254Q-R256K-L258F, and V14S-R40A-G59Y-G61 D-T64V-A66D-S70E-T117L-Q161 H-G175A-T177R-I178L-F180P-Y182A-R190L-S205G-F207T-V210I-S212D-F226L-A236P-Y239I-L249P-S252I-E254Q-R256K-L258F, wherein the positions are numbered by reference to the amino acid sequence of SEQ ID NO:2.

5. The cleaning or fabric conditioning composition claim 1, wherein the variant lipolytic enzyme has lipolytic activity on a polyester selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, polyethylene adipate) (PEA), and combinations thereof.

6. The cleaning or fabric conditioning composition of claim 1, wherein the variant lipolytic enzyme is contained in the composition in an amount of from 0.00001 to 1 wt. %, preferably in an amount of from 0.0001 to 0.5 wt. %, particularly preferably in an amount of from 0.001 to 0.1 wt. %.

7. The cleaning or fabric conditioning composition of claim 1, wherein the at least one additional ingredient comprises a performance polymer.

8. The cleaning or fabric conditioning composition of claim 7, wherein the performance polymer is an alkoxylated polyethylene imine.

9. The cleaning or fabric conditioning composition of claim 1, wherein the at least one additional ingredient comprises a complexing agent.

10. The cleaning or fabric conditioning agent of claim 9, wherein the complexing agent comprises citric acid/citrate, EDTA or a combination thereof.

11. The cleaning or fabric conditioning composition of claim 1, wherein the cleaning or fabric conditioning composition comprises at least one further ingredient selected from the group consisting of builders, bleaching agents, bleach activators, water-miscible organic solvents, sequestering agents, electrolytes, pH regulators, optical brighteners, graying inhibitors, foam regulators, dyes and fragrances and combinations thereof.

12. The cleaning or fabric conditioning composition of claim 1, wherein the cleaning or fabric conditioning composition has a pH of 7.0 to 11.0, as measured in 1 wt. % aqueous solution at 20° C.

13. The cleaning or fabric conditioning composition of claim 1, wherein the cleaning or fabric conditioning composition is present in liquid form.

14. The cleaning or fabric conditioning composition of claim 1, wherein the cleaning or fabric conditioning composition is in unit dose form.

15. A method of cleaning or conditioning a textile or fabric, comprising:
a) providing a composition comprising at least one variant lipolytic enzyme, wherein said variant lipolytic enzyme comprises an amino acid sequence having at least 70% identity to the full length amino acid sequence of SEQ ID NO:2, comprising the substitutions T064V-T117L-T177N/R-1178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161 H, G175A/E, F207L/T, V210I, Q227H, A236P, S244E, E254Q, and R256K, wherein the positions are numbered by reference to the amino acid sequence of SEQ ID NO:2, and wherein the variant has polyesterase activity; and at least one additional ingredient selected from the group consisting of complexing agents, surfactants, performance polymers and combinations thereof; and
b) contacting the textile or fabric with the composition,
wherein the textile or fabric comprises or consists of polyester, and wherein the polyester is preferably selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, polyethylene adipate) (PEA), and combinations thereof, more preferably selected from the group consisting of polyethylene terephthalate (PET), polytrimethylene terephthalate (PTT), polybutylene terephthalate (PBT), and combinations thereof.

16. Method for improving the thermophysiological properties of a textile or fabric comprising or consisting of polyester, the method comprising
a) providing a composition comprising at least one variant lipolytic enzyme, wherein said variant lipolytic enzyme comprises an amino acid sequence having at least 70% identity to the full length amino acid sequence of SEQ ID NO:2, comprising the substitutions T064V-T117L-T177N/R-I178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161H, G175A/E, F207L/T, V210I, Q227H, A236P, S244E, E254Q, and R256K, wherein the positions are numbered by reference to the amino acid sequence of SEQ ID NO:2, and wherein the variant has esterase activity; and
b) contacting the fabric or textile with the composition.

17. Method according to claim 16, wherein the thermophysiological properties comprise heat and moisture management, wear comfort or a combination thereof.

18. Method for increasing the hydrophilicity of a textile or fabric comprising or consisting of polyester, the method comprising
a) providing a composition comprising at least one variant lipolytic enzyme, wherein said variant lipolytic enzyme comprises an amino acid sequence having at least 70% identity to the full length amino acid sequence of SEQ ID NO:2, comprising the substitutions T064V-T117L-T177N/R-I178L-F180P-Y182A-R190L-S205G-S212D-F226L-Y239I-L249P-S252I-L258F, and further comprising at least one additional substitution selected from the group consisting of V014S, R040A/T, G059Y, G061D, A066D, S070E, Q161H, G175A/E, F207L/T, V210I, Q227H, A236P, S244E, E254Q, and R256K, wherein the positions are numbered by reference to the amino acid sequence of SEQ ID NO:2, and wherein the variant has esterase activity; and
b) contacting the fabric or textile with the composition.

19. The method according to claim 16, wherein the polyester is preferably selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, polyethylene adipate) (PEA), and combinations thereof, more preferably selected from the group consisting of polyethylene terephthalate (PET), polytrimethylene terephthalate (PTT), polybutylene terephthalate (PBT), and combinations thereof.
